(19) United States
(12) Patent Application Publication
(10) Pub. No.: US 2007/0060501 A1
(43) Pub. Date: Mar. 15, 2007

(54) Methods and therapies for potentiating a therapeutic action of an opioid receptor agonist and inhibiting and/or reversing tolerance to opioid receptor agonists

(76) Inventors: Khem Jhamandas, Kingston (CA); Brian Milne, Kingston (CA)

Correspondence Address: Licata & Tyrrell P.C., 66 East Main Street, Marlton, NJ, 08053, US

(21) Appl. No.: 11/515,301

(22) Filed: Aug. 30, 2006

Related U.S. Application Data
(60) Provisional application No. 60/753,958, filed on Dec. 23, 2005.
(60) Provisional application No. 60/712,545, filed on Aug. 30, 2005.

(51) Int. Cl. A61K 31/485; A61K 31/4745; A61K 31/551; A61K 38/33

(52) U.S. Cl. 514/2; 514/282; 514/220; 514/283

(57) ABSTRACT

Combination therapies of an opioid receptor agonist and an alpha-2 receptor antagonist in an amount effective to potentiate, but not antagonize, a therapeutic effect of the opioid receptor agonist are provided. Also provided are methods for use of these combination therapies in potentiating the therapeutic effects of opioid receptor agonists, inhibiting development of acute and/or chronic tolerance to opioid receptor agonists and treating conditions treatable by opioid receptor agonist therapy in a subject. In addition, a method for reversing opioid receptor agonist tolerance and/or restoring therapeutic effect of an opioid receptor agonist in a subject via administration of an alpha-2 receptor antagonist in an amount effective to potentiate, but not antagonize, the therapeutic effect of the opioid receptor agonist is provided.

### RELATED APPLICATIONS

This patent application claims the benefit of priority from U.S. Provisional Application Ser. No. 60/753,958, filed December 23, 2005, and U.S. Provisional Application Ser. No. 60/712,545, filed Aug. 30, 2005, teachings of each of which are herein incorporated by reference in their entirety.

### BACKGROUND OF THE INVENTION

Opioid drugs are indispensable in the clinical management of moderate to severe pain syndromes. Opioids are also used as cough suppressants, in the reduction and/or prevention of diarrhea, and in the treatment of pulmonary edema.

It is well-accepted that the potent analgesic actions of opioids result from interaction with specific receptors present on neurons in the brain, spinal cord and periphery. It is also recognized that there are multiple forms of these receptors. Cloning experiments have identified the existence of three distinct types of receptors, namely mu, delta and kappa. Each type of receptor is a distinct gene product and a 7 transmembrane G-protein coupled receptor (GPCR) (Kieffer et al., Trends in Pharmacol. Science 1999 20:19-26). These receptors are selectively targeted by endogenous opioid peptides and by highly selective agonistic or antagonistic ligands. In particular, endomorphins target mu receptors; enkephalins target delta receptors; and dynorphins target kappa receptors. Pharmacological evidence also suggests the existence of opioid receptor subtypes designated as mu₁ and mu₂, delta₁ and delta₂, and kappa₁, kappa₂, kappa₃ and kappa₄ (Pasternak and Standifer, Trends in Pharmacol. Science 1995 16:344-350). The molecular structure and/or origin of these opioid receptor subtypes is unclear although alternate processing of gene products (Rossi et al., FEBS Lett 1995 369:192-196; Pan et al., Mol. Pharmacol. 1999 396-403) and/or receptor oligomerization (Jordan and Devi, Nature 1999 399:697-700; George et al., J. Biol. Chem. 2000 275:26128-26135) have been suggested to provide a basis for additional receptor heterogeneity.

While opioids inhibit pain transmission by acting at different levels of the neuraxis, the dorsal spinal cord is recognized as a major site of their action. At this site, opioids inhibit activity of neurons signaling pain by presynaptic and postsynaptic actions. Presynaptically, opioids inhibit the release of several pain neurotransmitters including L-glutamate, calcitonin gene-related peptide (CGRP) and substance P from terminals of the high threshold primary afferents that are driven by the peripheral nociceptive inputs. This effect is attributable to the blockade of the voltage-gated N-type calcium channel (North et al., Proc. Natl Acad. Sci. USA 1987 84:5487-5491; Werz and McDonald, Neuropeptides 1984 5:253-256) regulating the calcium-dependent release of transmitters from nerve terminals. Postsynaptically, opioids hyperpolarize the projection neurons targeted by primary afferents by opening of potassium channels on these neurons. Activation of all opioid receptor types inhibits adenylyl cyclase activity, via a pertussis toxin (PTX)-sensitive mechanism.

The presynaptic and postsynaptic activity of nociceptive neurons is also modulated by several non-opioid receptors that operationally behave as opioid receptors. For example, activation of alpha-2 receptors on spinal nociceptive neurons reproduces the cellular and behavioral responses produced by opioid drugs (Ossipov et al., Anesthesiology 1990 73:1227-1235).

WO 03/099289 discloses a method for alleviating pain in a subject by administering a composition containing an alpha-adrenergic agonist and a selective alpha-2A antagonist.

However, while spinal administration of alpha-2 receptor agonists such as clonidine produce potent spinal analgesia, these agents, unlike opioids, produce significant cardiovascular effects by influencing the sympathetic outflow from the spinal cord. Further, like opioid receptor agonists, repeated exposure to spinal effects of alpha-2 receptor agonists can lead to the development of tolerance (Stevens et al., J. Pharm. Exp. Ther. 1998 244:63-70).

The development of tolerance, at least with respect to opioid receptor agonists, has been attributed to multiple factors (Jhamandas et al., Pain Res. Manag. 2000 5:25-32). Recent studies suggest that tolerance may result from the paradoxical stimulatory actions of opioids that are exerted at very low doses and that may progressively overwhelm the inhibitory effects contributing to analgesia (Crain and Shen, Trends in Pharm. Sci. 1990 11:177-81). The excitatory actions of opioids are blocked by opioid receptor antagonists (e.g. naloxone or naltrexone) when administered at ultra-low doses 50 to 100,000-fold lower than doses of opioid receptor antagonists blocking or inhibiting the classical opioid actions (Crain and Shen, Proc. Natl Acad. Sci USA 1995 92:10540-10544). Such ultra-low doses of the opioid receptor antagonist, naltrexone, paradoxically increase opioid analgesia, inhibit development of chronic opioid tolerance and reverse established tolerance (Powell et al., J. Pharmacol. Exp. Ther. 2002 300:588-596). The hypothesis underlying these actions is that the latent excitatory effects of an opioid produce hyperalgesia which is progressive and eventually overcomes the analgesia produced by classical opioid doses. However, clinical use of opioid receptor antagonists carries the risk of potential loss of the analgesic response.

Both non-selective adrenergic blockers phentolamine (alpha-1 and alpha-2 blocker) and propranolol (beta-1 and beta-2 blocker) and selective blockers prazoin (alpha-1 blocker) and metoprolol (beta-1 blocker) have been disclosed to suppress the development of tolerance to morphine analgesia in mice (Kihara, T. and Kaneto, H. Japan J. Pharmacol. 1986 42:419-423). Yohimbine (alpha-2 blocker), when administered at 5 mg/kg and 1 mg/kg, has been disclosed to delay, but not block, the development of tolerance to morphine (Kihara, T. and Kaneto, H. Japan J. Pharmacol. 1986 42:419-423). However, yohimbine is also disclosed to dose-dependently antagonize morphine analgesia in naive animals (Kihara, T. and Kaneto, H. Japan J. Pharmacol. 1986 42:419-423).

Various combination therapies for reducing the amount of opioid and/or alpha-2 receptor agonist required to provide analgesia have been described.

WO 98/38997 discloses use of levobupivacaine and an opioid or alpha-2 receptor agonist in a medicament for anesthesia and analgesia.

US2004/0254207 (published Dec. 16, 2004) and US2004/0092541 (published May 13, 2004) disclose N-acylated 4-hydroxyphenylamine derivatives for use in an analgesic composition also containing an opioid and caffeine or an opioid or non-opioid analgesic, respectively.

In recent years, functional interactions between spinal opioid receptors and alpha-2 receptors have been identified (Yaksh, T. I. Brain Res. 1979 160:180-185; Roerig et al. Brain Res. 1984 308:360-363; Wigdor, S. and Wilcox, G. J. Pharmacol. Exp. Ther. 1987 242:90-95; Stone et al. J. Neurosci. 1997 18:7157-7165).

The actions of alpha-2 receptor agonists are blocked by atipemazole and yohimbine. Atipemazole is a potent, selective and specific antagonist of both centrally and peripherally located alpha-2 adrenoceptors about 100 times more potent as a displacer of clonidine than yohimbine (Virtanen et al. Arch. Int. Pharmacodyn. 1989 297:190-204).

Browning et al. disclosed that the alpha-2 receptor agonist analgesic activity was antagonized only by alpha-2 receptor antagonists while the analgesic activity of morphine was antagonized by the opioid receptor antagonist naloxone, and by the alpha-2 receptor antagonist yohimbine (Br. J. Pharmacol. 1982 77:487-491). Based upon these studies in mouse and guinea pig ileum, Browning et al. showed that while yohimbine acts on alpha-2 receptors, it partially antagonizes the in vivo analgesic effects of opiates and weakly displaces the opioid radioligand binding. However, the opioid antagonist naloxone does not affect alpha-2 receptor agonist analgesia and opioid ligands do not displace alpha-2 receptor radioligand binding (Br. J. Pharmacol. 1982 77:487-491). In contrast, Kontinen and Kalso observed no cross antagonism between the mu-opioid and the alpha-2 adrenergic systems after administration of submaximal antinociceptive doses of morphine in the presence of the alpha-2 receptor antagonist, atipemazole and similar administration of dexmedetomidine in the presence of the opioid receptor antagonist, naloxone, in the rat tail flick or the hot plate test (Pharm. and Tox. 1995 76:368-370). Thus, unlike yohimbine, the alpha-2 receptor antagonist atipemazole neither antagonizes spinal morphine analgesia (Kontinen, V. K. and Kalso, E. A. (Pharm. and Tox. 1995 76:368-370)) nor does it show affinity for the opioid receptors (Virtanen et al. Arch. Int. Pharmacodyn. 1989 297:190-204).

A recent study indicates that the mu opioid receptors and the alpha-2 receptors can exist as a complex that is postulated to signal different responses, depending upon activation or blockade of either receptor (Jordan et al. Mol. Pharmacol. 2003 64:1317-1324). Data from this study suggests that mu opioid and alpha-2A adrenergic receptors can physically interact. Further, this interaction can be functionally enhanced by the addition of selective ligands for either system but not the addition of both ligands (Jordan et al. Mol. Pharmacol. 2003 64:1317-1324).

WO 2004/053099 (published Jun. 24, 2004) discloses a method for treating opioid drug addiction by administration of an effective amount of a variety of compounds, one of which is suggested to be an agonist or antagonist of an alpha-2 adrenergic receptor.

EP 0 906 757 (patent application published Apr. 7, 1999) discloses an analgesic composition comprising synergistically effective amounts of monoxidine, an alpha-2 receptor agonist (Kirch et al. J. Clin. Pharm. 1990 30:1088-1095), and an opioid analgesic agent.

### SUMMARY OF THE INVENTION

An aspect of the present invention is a composition comprising an opioid receptor agonist, in an amount effective to produce a therapeutic effect, and an alpha-2 receptor antagonist, in an amount effective to potentiate, but not antagonize, the therapeutic effect of the opioid receptor agonist. Compositions of the present invention provide useful therapeutic agents for management of pain including, but not limited to, acute and/or chronic post-surgical pain, obstetrical pain, acute and/or chronic inflammatory pain, pain associated with conditions such as multiple sclerosis and/or cancer, pain associated with trauma, pain associated with migraines, neuropathic pain, central pain and chronic pain syndrome of a non-malignant origin such as chronic back pain. Compositions of the present invention are also useful as cough suppressants, in reduction and/or prevention of diarrhea, in treatment of pulmonary edema and in alleviating physical dependence and/or addiction to opioid receptor agonists.

Another aspect of the present invention is a method for potentiating a therapeutic effect of an opioid receptor agonist which comprises administering to a subject in combination with an opioid receptor agonist an alpha-2 receptor antagonist in an amount effective to potentiate, but not antagonize, the therapeutic effect of the opioid receptor agonist.

Another aspect of the present invention is a method for potentiating a biological action of an endogenous opioid receptor agonist in a subject which comprises administering to the subject an alpha-2 receptor antagonist in an amount effective to potentiate, but not antagonize, the biological action of the endogenous opioid receptor agonist.

Another aspect of the present invention is a method for inhibiting development of acute tolerance to a therapeutic action of an opioid receptor agonist in a subject which comprises administering to a subject in combination with an opioid receptor agonist an alpha-2 receptor antagonist in an amount effective to potentiate, but not antagonize, the therapeutic effect of the opioid receptor agonist.

Another aspect of the present invention is a method for inhibiting development of chronic tolerance to a therapeutic action of an opioid receptor agonist in a subject which comprises administering to a subject in combination with an opioid receptor agonist an alpha-2 receptor antagonist in an amount effective to potentiate, but not antagonize, the therapeutic effect of the opioid receptor agonist.

Another aspect of the present invention is a method for reversing tolerance to a therapeutic action of an opioid receptor agonist and/or restoring therapeutic potency of an opioid receptor agonist in a subject tolerant to a therapeutic action of an opioid receptor agonist which comprises administering an alpha-2 receptor antagonist to a subject receiving an opioid receptor agonist in an amount effective to potentiate, but not antagonize, the therapeutic effect of the opioid receptor agonist.

Another aspect of the present invention is a method for treating a subject suffering from a condition treatable with an opioid receptor agonist comprising administering to the subject an opioid receptor agonist in an amount effective to produce a therapeutic effect and an alpha-2 receptor antagonist in an amount effective to potentiate, but not antagonize, the therapeutic effect of the opioid receptor agonist.

The above methods are useful for treating subjects suffering from conditions including, but not limited to, pain, coughs, diarrhea, pulmonary edema and addiction to opioid receptor agonists. It is understood that such treatment may also be commenced prior to such suffering (i.e., prophylactically, when the subject is at risk for such suffering).

Yet a further aspect of the present invention in each of the above methods is that the opioid receptor antagonist is administered or formulated in an amount which potentiates, but does not antagonize, the therapeutic effect of the opioid receptor agonist, and that the amount of the opioid receptor antagonist, alone or in combination with the opioid receptor agonist, does not elicit a substantial undesirable side effect.

### BRIEF DESCRIPTION OF THE FIGURES

FIGS. 1A and 1B are line graphs showing the effects of the alpha-2 receptor antagonist atipemazole at inhibiting analgesia by the alpha-2 receptor agonist clonidine in a tail flick test (FIG. 1A) and a paw pressure test (FIG. 1B) in rats. Clonidine was administered intrathecally at 200 nmoles which is equal to 53.2 micrograms per rat. Rats were co-administered atipemazole intrathecally at 0 micrograms/rat (open circle), 1 microgram/rat (filled square), 5 micrograms/rat (filled triangle), and 10 micrograms/rat (inverted filled triangle).

FIGS. 2A and 2B are line graphs showing the effects of the alpha-2 receptor antagonist atipemazole administered at a dose ineffective at causing alpha-2 receptor blockade on acute tolerance to the analgesic actions of spinal morphine in the tail flick test (FIG. 2A) and paw pressure test (FIG. 2B) in rats. In this study, acute tolerance was produced by delivering three intrathecal successive injections (depicted by vertical arrows) of morphine (15 μg) at 90 minute intervals (depicted by open circles). A second group of rats received a combination of morphine (15 μg) and a fixed dose of atipemazole (0.8 ng) (depicted by filled circles). The effects of atipemazole alone (0.8 ng)(depicted as filled triangles) and normal saline (20 μl)(depicted as open squares) were also evaluated by injecting these at 90 minute intervals.

FIGS. 3A and 3B are cumulative dose-response curves (DRCs) for the acute analgesic action of intrathecal morphine, in the four treatment groups of FIGS. 2A and 2B, derived 24 hours after the first morphine injection. Rats administered morphine (15 μg) alone are depicted by open circles. Rats administered morphine (15 μg) and atipemazole (0.8 ng) are depicted by filled circles. Rats administered atipemazole (0.8 ng) alone are depicted by open triangles. Rats administered saline (20 μl) are depicted by inverted open triangles.

FIGS. 4A and 4B are bar graphs showing the ED₅₀ values (effective dose in 50% of the animals), an index of potency, derived from the cumulative dose-response curves of FIGS. 3A and 3B, respectively. Rats administered morphine (15 μg) alone are depicted by the horizontal lined bar. Rats administered morphine (15 μg) and atipemazole (0.8 ng) are depicted by the horizontal and vertical lined bar. Rats administered atipemazole (0.8 ng) alone are depicted by the vertical lined bar. Rats administered saline (20 μl) are depicted by the unfilled bar.

FIGS. 5A and 5B are line graphs showing the effects of administration of the alpha-2 receptor antagonist atipemazole, at doses ineffective at causing alpha-2 receptor blockade, on the acute morphine analgesia in the tail flick (FIG. 5A) and paw pressure test (FIG. 5B) in rats. Rats administered morphine (15 μg) alone are depicted by open circles. Rats administered morphine (15 μg) and atipemazole at 0.8 ng are depicted by filled triangles. Rats administered morphine (15 μg) and atipemazole at 0.08 ng are depicted by inverted filled triangles. Rats administered atipemazole alone at 0.8 ng are depicted by open triangles.

FIGS. 6A and 6B are line graphs showing the effects of spinal administration of the alpha-2 receptor antagonist atipemazole, at doses ineffective at causing alpha-2 receptor blockade, on the chronic morphine tolerance induced by daily opioid administration at 30 minutes after daily drug administration in the tail flick (FIG. 6A) and paw pressure test (FIG. 6B) in rats. Rats administered morphine (15 μ/day) alone are depicted by open circles. Rats administered morphine (15 μg) and atipemazole at 0.8 ng/day are depicted by filled triangles. Rats administered morphine (15 μg/day) and atipemazole at 0.08 ng/day are depicted by inverted filled triangles. Rats administered atipemazole alone at 0.8 ng/day are depicted by open triangles.

FIGS. 7A and 7B are line graphs showing the effects of spinal administration of the alpha-2 receptor antagonist atipemazole at doses ineffective at causing alpha-2 receptor blockade on the chronic morphine tolerance induced by daily opioid administration at 60 minutes after daily drug administration in the tail flick (FIG. 7A) and paw pressure test (FIG. 7B) in rats. Rats administered morphine (15 μg/day) alone are depicted by open circles. Rats administered morphine (15 μg/day) and atipemazole at 0.8 ng/day are depicted by filled triangles. Rats administered morphine (15 μg/day) and atipemazole at 0.08 ng/day are depicted by inverted filled triangles. Rats administered atipemazole alone at 0.8 ng/day are depicted by open triangles.

FIGS. 8A and 8B are cumulative dose-response curves for the analgesic action of morphine, in the four treatment groups of FIGS. 7A and 7B, derived on day 6, i.e. 24 hours after cessation of the 5 day chronic drug treatment. Rats administered morphine (15 μg/day) alone are depicted by open circles. Rats administered morphine (15 μ/day) and atipemazole at 0.8 ng/day are depicted by filled triangles. Rats administered morphine (15 μg/day) and atipemazole at 0.08 ng/day are depicted by filled inverted triangles. Rats administered atipemazole alone at 0.8 ng/day are depicted by open triangles.

FIGS. 9A and 9B are bar graphs showing the ED₅₀ values, an index of potency, derived from the cumulative dose-response curves of FIGS. 8A and 8B, respectively. Rats administered morphine alone are depicted by the unfilled bar. Rats administered morphine and atipemazole at 0.8 ng are depicted by the right-hatch lined bar. Rats administered morphine and atipemazole at 0.08 ng are depicted by the left-hatch lined bar. Rats administered atipemazole alone at 0.8 ng are depicted by the horizontal and vertical lined bar.

FIGS. 10A and 10B are line graphs illustrating the time course of the analgesic responses, in the rat tail flick (FIG. 10A) and paw pressure test (FIG. 10B), produced by the atipemazole-morphine combination at conclusion of a chronic treatment period (day 5). Rats administered morphine (15 μg) alone are depicted by open circles. Rats administered morphine (15 μg) and atipemazole at 0.8 ng are depicted by filled triangles. Rats administered morphine (15 μg) and atipemazole at 0.08 ng are depicted by inverted filled triangles. Rats administered atipemazole at 0.8 ng alone are depicted by open triangles.

FIGS. 11A and 11B are line graphs demonstrating the reversal of tolerance to the morphine induced after 5 days of treatment in the rat tail flick (FIG. 11A) and paw pressure test (FIG. 11B) following administration of atipemazole. Rats administered morphine alone (15 μg) for ten days are depicted by open circles. Rats administered morphine (15 μg) for 10 days and atipemazole at 0.8 ng beginning at day 6 for 5 days are depicted by filled circles. Nociceptive testing was performed at 30 minutes post daily injection.

FIGS. 12A and 12B are line graphs demonstrating the reversal of tolerance to the morphine induced after 5 days of treatment in the rat tail flick (FIG. 12A) and paw pressure test (FIG. 12B) following administration of atipemazole. Rats administered morphine alone (15 μg) for ten days are depicted by open circles. Rats administered morphine (15 μg) for 10 days and atipemazole at 0.8 ng beginning at day 6 for 5 days are depicted by filled circles. Nociceptive testing was performed at 60 minutes post daily in injection. Vertical arrows indicate time of dose-response curves depicted in FIGS. 13A and 13B.

FIGS. 13A and 13B are line graphs showing the cumulative dose-response curves for intrathecal morphine obtained in the two animal groups represented in FIGS. 12 A and 12B. Rats administered morphine (15 μg) alone for ten days are depicted by open circles. Rats administered morphine (15 μg) for 10 days and atipemazole at 0.8 ng beginning at day 6 for 5 days are depicted by filled circles.

FIGS. 14A and 14B are bar graphs showing the ED₅₀ values, an index of potency, derived from the cumulative dose-response curves of FIGS. 13A and 13B, respectively. Rats administered morphine (15 μg) alone are depicted by the unfilled bar. Rats administered morphine (15 μg) for 10 days and atipemazole at 0.8 ng beginning at day 6 for 5 days are depicted by the vertical lined bar.

FIGS. 15A and 15B are line graphs showing the antagonistic effects of the alpha-2 receptor antagonist yohimbine at inhibiting spinal analgesia by the alpha-2 receptor agonist clonidine in the tail flick test (FIG. 15A) and paw pressure test (FIG. 15B) in rats. Rats were administered clonidine (13.3 μg) intrathecally alone (open circles), yohimbine (30 μg) intrathecally alone (open triangles), or clonidine (13.3 μg) and yohimbine (30 μg) intrathecally (filled squares).

FIGS. 16A and 16B are line graphs showing the antagonistic effects of the alpha-2 receptor antagonist yohimbine at inhibiting spinal morphine analgesia in the tail flick test (FIG. 16A) and paw pressure test (FIG. 16B). Rats were administered morphine (15 μg) intrathecally alone (open circles), yohimbine (30 μg) intrathecally alone (open triangles), or morphine (15 μg) and yohimbine (30 μg) intrathecally (filled squares).

FIG. 17A and FIG. 17B are line graphs showing the effects of administration of the alpha-2 receptor antagonist yohimbine, at doses ineffective at causing alpha-2 receptor blockade, on analgesia produced by a single spinal dose of morphine in the tail flick (FIG. 17A) and paw pressure test (FIG. 17B) in rats. Rats administered morphine (15 μg) alone are depicted by open circles. Rats administered morphine (15 μg) and yohimbine (0.024 ng) are depicted by filled squares. Rats administered morphine (15 μg) and yohimbine (2.4 ng) are depicted by inverted filled triangles. Rats administered morphine (15 μg) and yohimbine (5 ng) are depicted by filled diamonds. Rats administered yohimbine alone (0.024 ng) are depicted by open squares. Rats administered yohimbine alone at 2.4 ng are depicted by open inverted triangles.

FIGS. 18A and 18B are line graphs showing the effects of the alpha-2 receptor antagonist yohimbine administered at a dose ineffective at causing alpha-2 receptor blockade on acute tolerance to the analgesic actions of spinal morphine in the tail flick test (FIG. 18A) and paw pressure test (FIG. 18B) in rats. In this study, acute tolerance was produced by delivering three intrathecal successive injections (indicated by arrowheads) of morphine (15 μg) at 90 minute intervals (depicted by open circles). Other groups of rats received a combination of morphine (15 μg) and a fixed dose of yohimbine of 0.0048 ng (depicted by filled squares), 0.024 ng (filled triangles), or 0.24 ng (inverted filled triangles). The effects of yohimbine alone (0.024 ng; depicted as open triangles) and normal saline (20 μl; depicted as Xs) were also evaluated by injecting these at 90 minute intervals.

FIG. 19A and FIG. 19B are cumulative dose-response curves (DRCs) for the acute analgesic action of intrathecal morphine, in the six treatment groups of FIGS. 18A and 18B, respectively, derived 24 hours after the first morphine injection. Rats administered morphine (15 μg) alone are depicted by open circles. Rats administered morphine (15 μg) and yohimbine at 0.0048 ng are depicted by filled squares. Rats administered morphine (15 μg) and yohimbine at 0.024 ng are depicted by filled triangles. Rats administered morphine (15 μg) and yohimbine at 0.24 ng are depicted by filled inverted triangles. Rats administered yohimbine (0.024 ng) alone are depicted by open triangles. Rats administered saline are depicted by Xs.

FIGS. 20A and 20B are bar graphs showing the ED₅₀ values (effective dose in 50% of the animals), an index of potency, derived from the cumulative dose-response curves of FIGS. 19A and 19B, respectively. Rats administered morphine (15 μg) alone are depicted by the dotted bar. Rats administered morphine (15 μg) and yohimbine at 0.0048 ng are depicted by the left hatched bar. Rats administered morphine (15 μg) and yohimbine at 0.024 ng are depicted by the right hatched bar. Rats administered morphine (15 μg) and yohimbine at 0.24 ng are depicted by the vertical lined bar. Rats administered yohimbine (0.024 ng) alone are depicted by the horizontal lined bar. Rats administered saline are depicted by the unfilled bar.

FIGS. 21A and 21B are line graphs showing the antagonistic effects of the alpha-2 receptor antagonist mirtazapine at inhibiting spinal analgesia by the alpha-2 receptor agonist clonidine in the tail flick test (FIG. 21A) and paw pressure test (FIG. 21B) in rats. Rats were administered clonidine (13.3 μg) intrathecally alone (open squares) or clonidine (13.3 μg) and mirtazapine (2μg) intrathecally (filled squares).

FIGS. 22A and 22B are line graphs showing the effects of administration of the alpha-2 receptor antagonist mirtazapine, at doses ineffective at causing alpha-2 receptor blockade, on analgesia produced by a single spinal dose of morphine in the tail flick (FIG. 22A) and paw pressure test (FIG. 22B) in rats. Rats were administered morphine (15 μg) intrathecally alone (open circles), morphine (15 μg) and mirtazapine (0.02 ng) intrathecally (filled triangle), or morphine (15 μg) and mirtazapine (0.2 ng) intrathecally (filled, inverted triangle).

FIG. 23A and 23B are line graphs showing the effects of the alpha-2 receptor antagonist mirtazapine administered at a dose ineffective at causing alpha-2 receptor blockade on acute tolerance to the analgesic actions of spinal morphine in the tail flick test (FIG. 23A) and paw pressure test (FIG. 23B) in rats. In this study, acute tolerance was produced by delivering three intrathecal successive injections (indicated by arrowheads) of morphine (15 μg) at 90 minute intervals (depicted by open circles). Another group of rats received a combination of morphine (15 μg) and a fixed dose of mirtazapine of 0.02 ng (depicted by filled triangles). The effects of normal saline 20 μl; depicted as Xs) injected at 90 minute intervals were also evaluated.

FIG. 24A and FIG. 24B are cumulative dose-response curves (DRCs) for the acute analgesic action of intrathecal morphine, in the three treatment groups of FIGS. 23A and 23B, respectively, derived 24 hours after the first morphine injection. Rats administered morphine (15 μg) alone are depicted by open circles. Rats administered morphine (15 μg) and mirtazapine at 0.02 ng are depicted by filled triangles. Rats administered saline are depicted by Xs.

FIGS. 25A and 25B are bar graphs showing the ED₅₀ values (effective dose in 50% of the animals), an index of potency, derived from the cumulative dose-response curves of FIGS. 24A and 24B, respectively. Rats administered morphine (15 μg) alone are depicted by the dotted bar. Rats administered morphine (15 μg) and mirtazapine at 0.02 ng are depicted by the horizontally lined bar. Rats administered saline (20 μl) are depicted by the unfilled bar.

FIG. 26A and 26B are line graphs showing cumulative morphine dose-response curves obtained 24 hours after pretreatment with a single mirtazapine dose followed by repeated morphine administration in the tail flick test (FIG. 26A) and paw pressure test (FIG. 26B). In this study, acute tolerance was produced by delivering three intrathecal successive injections of morphine (15 μg) at 90 minute intervals (depicted by open circles). Other groups of rats received three intrathecal successive injections of morphine (15 μg) at 90 minute intervals and a single dose of mirtazapine (0.02 ng) (depicted by filled triangles) 30 minutes prior to morphine administration or three intrathecal successive injections of saline (20 μl) at 90 minute intervals and a single dose of mirtazapine (0.02 ng) (depicted by open triangles) prior to saline administration. The effects of normal saline (20 μl; depicted as Xs) injected at 90 minute intervals were also evaluated.

FIGS. 27A and 27B are bar graphs showing the ED₅₀ values (effective dose in 50% of the animals), an index of potency, derived from the cumulative dose-response curves of FIGS. 26A and 26B, respectively. Rats administered morphine (15 μg) alone are depicted by the dotted bar. Rats administered saline (20 μl) and mirtazapine at 0.02 ng are depicted by the horizontally lined bar. Rats administered morphine (15 μg) and mirtazapine at 0.02 ng are depicted by the vertically lined bar. Rats administered saline (20 μl) are depicted by the unfilled bar.

FIGS. 28A and 28B are line graphs showing the antagonistic effects of the alpha-2 receptor antagonist idazoxan at inhibiting spinal analgesia by the alpha-2 receptor agonist clonidine in the tail flick test (FIG. 28A) and paw pressure test (FIG. 28B) in rats. Rats were administered clonidine (13.3 μg) intrathecally alone (open squares), idazoxan (10 μg intrathecally alone (open diamonds), clonidine (13.3 μg) and idazoxan (10 μg) intrathecally (filled squares), or saline (20 μl; depicted by Xs).

FIGS. 29A and 29B are line graphs showing the effects of administration of the alpha-2 receptor antagonist idazoxan, at doses ineffective at causing alpha-2 receptor blockade, on analgesia produced by a single spinal dose of morphine in the tail flick (FIG. 29A) and paw pressure test (FIG. 29B) in rats. Rats were administered morphine (15 μg) intrathecally alone (open circles), morphine (15 μg) and idazoxan (0.08 ng) intrathecally (filled circles), or saline (20 μl; depicted as Xs).

FIG. 30A and 30B are line graphs showing the effects of the alpha-2 receptor antagonist idazoxan administered at a dose ineffective at causing alpha-2 receptor blockade on acute tolerance to the analgesic actions of spinal morphine in the tail flick test (FIG. 30A) and paw pressure test (FIG. 30B) in rats. In this study, acute tolerance was produced by delivering three intrathecal successive injections of morphine (15 μg) at 90 minute intervals (depicted by open circles). Other groups of rats received idazoxan alone at 0.016 ng (depicted by open triangles) or 0.08 ng (depicted by inverted open triangles), or a combination of morphine (15 μg) and a fixed dose of idazoxan of 0.008 ng (depicted by inverted filled triangles), 0.016 ng (depicted by filled triangles) or 0.08 ng (depicted by filled diamonds). The effects of normal saline (20 μl; depicted as Xs) injected at 90 minute intervals were also evaluated.

FIG. 31A and FIG. 31B are cumulative dose-response curves (DRCs) for the acute analgesic action of intrathecal morphine, in the 7 treatment groups of FIGS. 30A and 30B, respectively, derived 24 hours after the first morphine injection. Rats administered morphine (15 μg) alone are depicted by open circles. Rats administered idazoxan alone at 0.016 ng are depicted by open triangles. Rats administered idazoxan alone at 0.008 ng are depicted by inverted open triangles). Rats administered a combination of morphine (15 μg) and a fixed dose of idazoxan of 0.008 ng are depicted by inverted filled triangles. Rats administered a combination of morphine (15 μg) and a fixed dose of idazoxan of 0.016 ng are depicted by filled triangles. Rats administered a combination of morphine (15 μg) and a fixed dose of idazoxan of 0.08 ng are depicted by filled diamonds). Rats administered saline are depicted by Xs.

FIGS. 32A and 32B are bar graphs showing the ED₅₀ values (effective dose in 50% of the animals), an index of potency, derived from the cumulative dose-response curves of FIGS. 31A and 31B, respectively. Rats administered morphine (15 μg) alone are depicted by the dotted bar. Rats administered idazoxan alone at 0.008 ng are depicted by the horizontally line bar. Rats administered idazoxan alone at 0.016 ng are depicted by the vertically lined bar. Rats administered a combination of morphine (15 μg) and a fixed dose of idazoxan of 0.008 ng are depicted by the horizontally and vertically lined bar. Rats administered a combination of morphine (15 μg) and a fixed dose of idazoxan of 0.016 ng are depicted by the right-hatch lined bar. Rats administered a combination of morphine (15 μg) and a fixed dose of idazoxan of 0.08 ng are depicted by the left-hatch lined bar. Rats administered saline (20 μl) are depicted by Xs. Rats administered saline are depicted by the unfilled bar.

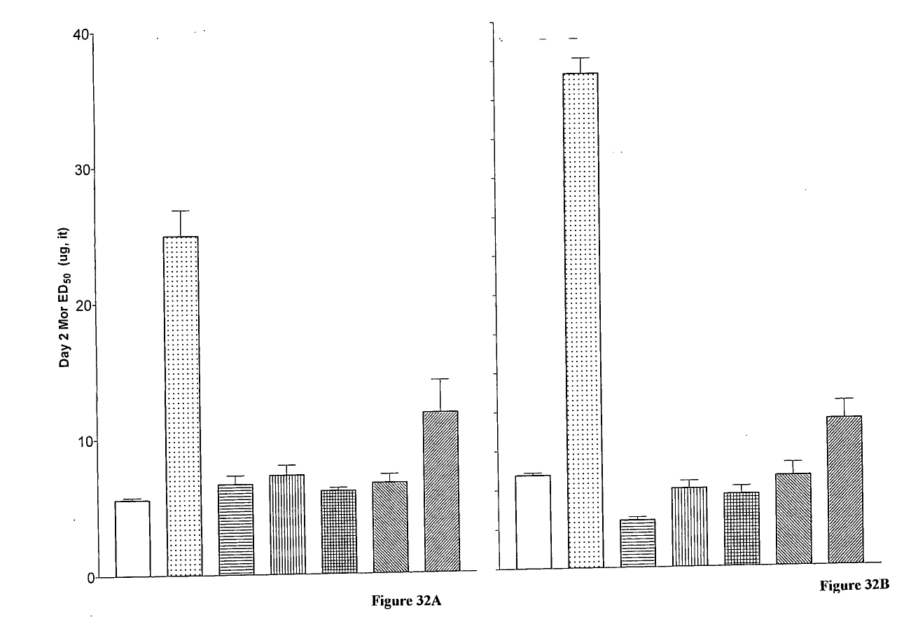

### DETAILED DESCRIPTION OF THE INVENTION

It has now been found that administration of an ultra-low dose of an alpha-2 receptor antagonist potentiates opioid receptor agonist analgesia and inhibits, delays or reduces the development of acute or chronic tolerance to opioid receptor agonists. The present invention provides new combination therapies for potentiating therapeutic activities of an opioid receptor agonist and inhibiting, delaying or reducing development of and/or reversing, at least partially, chronic and/or acute tolerance to an opioid receptor agonist involving co-administration of an opioid receptor agonist with an alpha-2 receptor antagonist. An aspect of the present invention thus relates to compositions comprising an opioid receptor agonist and an ultra-low dose of an alpha-2 receptor antagonist. Another aspect of the present invention relates to methods for potentiating a therapeutic action of an opioid receptor agonist and/or effectively inhibiting, delaying or reducing the development of acute as well as chronic tolerance to a therapeutic action of an opioid receptor agonist by co-administering the opioid receptor agonist with an ultra-low dose of an alpha-2 receptor antagonist. The new combination therapies of the present invention are expected to be useful in optimizing the use of opioid drugs in various applications including but not limited to: pain management, e.g. management of acute or chronic post-surgical pain, obstetrical pain, acute or chronic inflammatory pain, pain associated with conditions such as multiple sclerosis or cancer, pain associated with trauma, pain associated with migraines, neuropathic pain, and central pain; management of chronic pain syndrome of a non-malignant origin such as chronic back pain; cough suppression; reducing and/or preventing diarrhea; treating pulmonary edema; and alleviating addiction to opioid receptor agonists. In a preferred embodiment, the combination therapies of the present invention are used in pain management.

Alpha-2 receptor antagonists useful in the combination therapies and methods of the present invention include any compound that partially or completely reduces, inhibits, blocks, inactivates and/or antagonizes the binding of an alpha-2 receptor agonist to its receptor to any degree and/or the activation of an alpha-2 receptor to any degree. Thus, the term alpha-2 receptor antagonist is also meant to include compounds that antagonize the agonist in a competitive, irreversible, pseudo-irreversible and/or allosteric mechanism. In addition, the term alpha-2 receptor antagonist includes compounds at ultra-low dose that increase, potentiate and/or enhance the therapeutic and/or analgesic potency and/or efficacy of opioid receptor agonists, while at such doses do not demonstrate a substantial or significant antagonism of an alpha-2 receptor agonist. Examples of alpha-2 receptor antagonists useful in the combination therapies and methods of the present invention include, but are in no way limited to atipemazole (or atipamezol), fipamazole (fluorinated derivative of atipemazole), mirtazepine (or mirtazapine), eferoxan, idozoxan (or idazoxan), Rx821002 (2-methoxy-idozoxan), rauwolscine, MK 912, SKF 86466, SKF 1563 and yohimbine. Additional examples of agents which exhibit some alpha 2 and/or alpha 1 receptor antagonistic activity and thus may be useful in the present invention include, but are not limited to, venlafaxine, doxazosin, phentolamine, dihydroergotamine, ergotamine, phenothiazines, phenoxybenzamine, piperoxane, prazosin, tamsulosin, terazosin, and tolazoline. The alpha-2 receptor antagonist is included in the compositions and administered in the methods of the present invention at an ultra-low dose.

Compositions of the present invention as well as methods described herein for their use may comprise an ultra-low dose of more than one alpha-2 receptor antagonist alone, or more than one alpha-2 receptor antagonist at an ultra-low dose in combination with one or more opioid receptor agonists.

The alpha-2 receptor antagonist is included in the compositions and administered in the methods of the present invention at an ultra-low dose. By ultra-low dose as used herein it is meant an amount of alpha-2 receptor antagonist that potentiates, but does not antagonize, a therapeutic effect of the opioid receptor agonist. Thus, in one embodiment, by the term “ultra-low dose” it is meant an amount of the alpha-2 receptor antagonist lower than that established by those skilled in the art to significantly block or inhibit alpha-2 receptor activity.

As used herein, the term “amount” is intended to refer to the quantity of alpha-2 receptor antagonist and/or opioid receptor agonist administered to a subject. The term “amount” encompasses the term “dose” or “dosage”, which is intended to refer to the quantity of alpha-2 receptor antagonist and/or opioid receptor agonist administered to a subject at one time or in a physically discrete unit, such as, for example, in a pill, injection, or patch (e.g., transdermal patch). The term “amount” also encompasses the quantity of alpha-2 receptor antagonist and/or opioid receptor agonist administered to a subject, expressed as the number of molecules, moles, grams, or volume per unit body mass of the subject, such as, for example, mol/kg, mg/kg, ng/kg, ml/kg, or the like, sometimes referred to as concentration administered.

In accordance with the invention, administration to a subject of a given amount of alpha-2 receptor antagonist and/or opioid receptor agonist results in an effective concentration of the antagonist and/or agonist in the subject's body. As used herein, the term “effective concentration” is intended to refer to the concentration of alpha-2 receptor antagonist and/or opioid receptor agonist in the subject's body (e.g., in the blood, plasma, or serum, at the target tissue(s), or site(s) of action) capable of producing a desired therapeutic effect. The effective concentration of alpha-2 receptor antagonist and/or opioid receptor agonist in the subject's body may vary among subjects and may fluctuate within a subject over time, depending on factors such as, but not limited to, the condition being treated, genetic profile, metabolic rate, biotransformation capacity, frequency of administration, formulation administered, elimination rate, and rate and/or degree of absorption from the route/site of administration. For at least these reasons, for the purpose of this disclosure, administration of alpha-2 receptor antagonist and/or opioid receptor agonist is conveniently provided as amount or dose of alpha-2 receptor antagonist or opioid receptor agonist. The amounts, dosages, and dose ratios provided herein are exemplary and may be adjusted, using routine procedures such as dose titration, to provide an effective concentration.

In one embodiment the amount of alpha-2 receptor antagonist administered potentiates, but does not antagonize, a therapeutic effect of an opioid receptor agonist. Thus, the effective concentration of an alpha-2 receptor antagonist is a concentration in the body which potentiates the therapeutic action of an opioid receptor agonist. Preferably, the amount of alpha-2 receptor antagonist administered potentiates the therapeutic action of the opioid receptor agonist without the amount of the alpha-2 receptor antagonist, alone or in combination with the opioid receptor agonist, eliciting a substantial undesirable side effect.

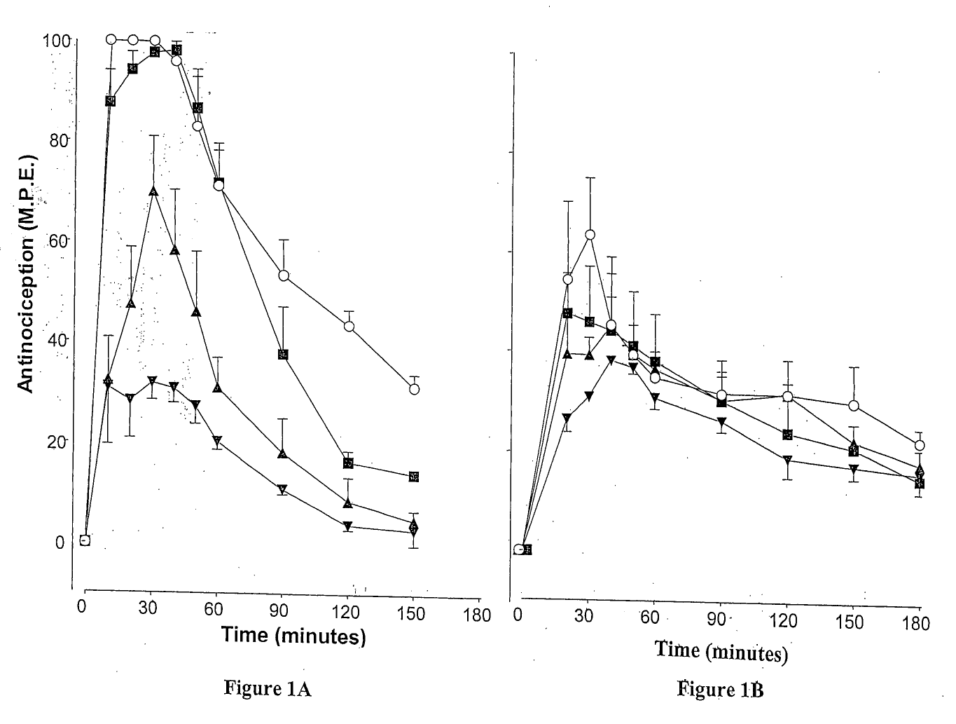

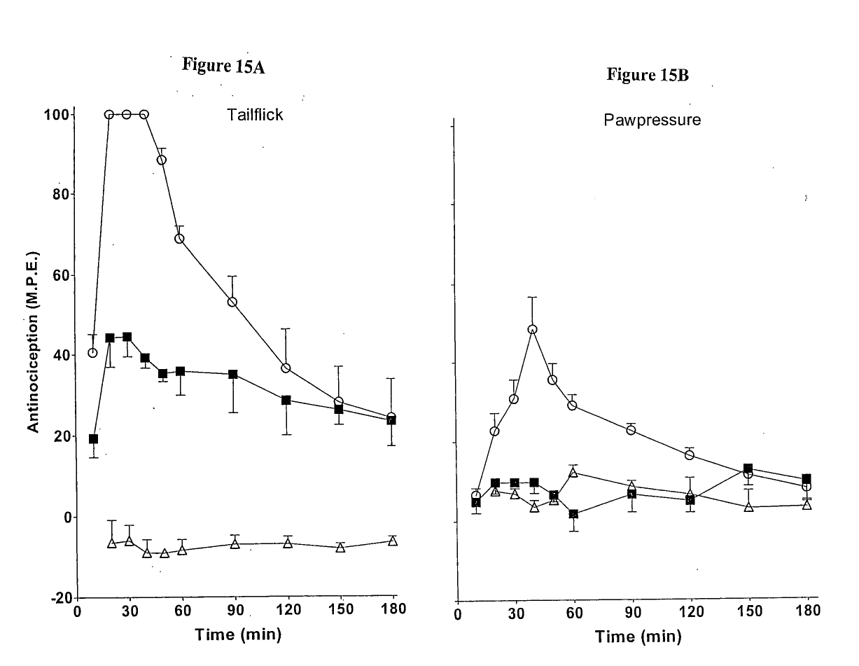

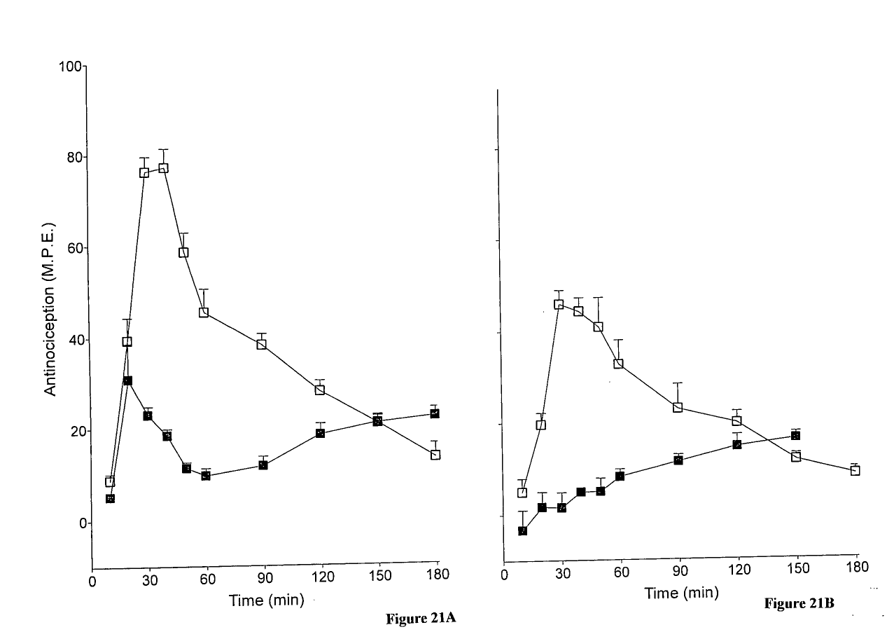

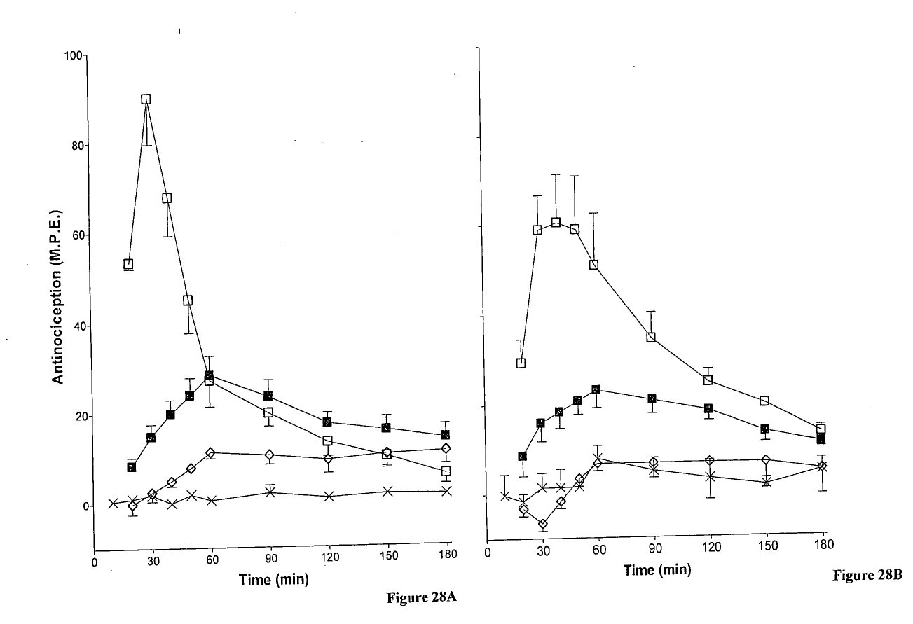

For example, in one embodiment, an ultra-low dose of alpha-2 receptor antagonist is an amount ineffective at alpha-2 receptor blockade as measured in experiments such as set forth in FIGS. 1A and 1B, FIGS. 15A and 15B, FIGS. 21A and 21B and FIGS. 28A and 28B. As will be understood by the skilled artisan upon reading this disclosure, however, other means for measuring alpha-2 receptor antagonism can be used. Based upon these experiments, ultra-low doses of atipemazole which potentiate the analgesic action of the opioid morphine were identified as being 12,000-fold to 120,000-fold lower than the dose producing a blockade of the spinal alpha-2 receptors, as evidenced by antagonism of intrathecal clonidine (alpha-2 agonist) analgesia (FIG. 1A and FIG. 1B). Ultra-low doses of yohimbine which potentiate the analgesic action of the opioid morphine were identified as being 6,000 to 6,250,000-fold lower than the dose producing a blockade of the spinal alpha-2 receptors, as evidenced by antagonism of intrathecal clonidine (alpha-2 agonist) analgesia (FIG. 15A and 15B). Ultra-low doses of mirtazapine which potentiate the analgesic action of the opioid morphine were identified as 10,000 to 100,000-fold lower than the dose producing a blockade of the spinal alpha-2 receptors, as evidenced by antagonism of intrathecal clonidine (alpha-2 agonist) analgesia (FIG. 21A and 21B). Ultra-low doses of idazoxan which potentiate the analgesic action of the opioid morphine were identified as 125,000 to 1,250,000-fold lower than the dose producing a blockade of the spinal alpha-2 receptors, as evidenced by antagonism of intrathecal clonidine (alpha-2 agonist) analgesia (FIG. 28A and 28B). Ultra-low doses useful in the present invention for other alpha-2 receptor antagonists as well as other therapeutic actions of opioids can be determined routinely by those skilled in the art in accordance with the known effective concentrations as alpha-2 receptor blockers and the methodologies described herein for atipemazole, yohimbine, mirtazepine and/or idazoxan. In general, however, by “ultra-low” it is meant a dose at least 1,000- to 6,250,000-fold lower that the maximal dose producing a blockade of alpha-2 receptors.

An exemplary embodiment of an “ultra-low dose” is an amount of alpha-2 receptor antagonist which is significantly less than the amount of opioid receptor agonist to be administered. Thus, in this embodiment, the ultra-low dose of alpha-2 receptor antagonist is expressed as a ratio with respect to the dose of opioid receptor agonist administered or to be administered. In this embodiment a preferred ratio for an ultra-low dose is a ratio of 1:1,000, 1:10,000, 1:100,000 or 1:1,000,000 or any ratio in between of alpha-2 receptor antagonist to opioid receptor agonist.

In another embodiment, the alpha-2 receptor antagonist and opioid receptor agonist are administered to a subject in amounts that result in relative ratios of amounts or effective concentrations within the blood, plasma, serum, or at the target tissue(s), or site(s) of action of 1:1,000, 1:10,000, 1:100,000, or 1:1,000,000 or any ratio in between.

Another exemplary embodiment of an “ultra-low” dose is an amount or ratio which potentiates the therapeutic action of the opioid receptor agonist without the amount of alpha-2 receptor antagonist, alone or in combination with the opioid receptor agonist, eliciting a substantial undesirable side effect.

By “substantial undesirable side effect” as used herein it is meant a response in a subject to the alpha-2 receptor antagonist other than potentiating the therapeutic action of the opioid receptor agonist which can not be controlled in the subject and/or endured by the subject and/or could result in discontinued treatment of the subject with the combination therapies and methods of the present invention.

Examples of such side effects include, but are not limited to, tolerance, dependence, addiction, sedation, euphoria, dysphoria, memory impairment, hallucination, depression, headache, hyperalgesia, constipation, insomnia, body aches and pains, change in libido, respiratory depression and/or difficulty breathing, nausea and vomiting, pruritus, dizziness, fainting (i.e. syncope), nervousness and/or anxiety, irritability, psychoses, tremors, changes in heart rhythm, decrease in blood pressure, elevated in blood pressure, elevated heart rate, risk of heart failure, temporary muscle paralysis and diarrhea.

Opioid receptor agonists useful in the combination therapies and methods of the present invention include any compound (either endogenous or exogenous to the subject) that binds to and/or activates and/or agonizes an opioid receptor to any degree and/or stabilizes the opioid receptor in an active or inactive conformation. Thus, by the term opioid receptor agonist it is meant to include partial agonists, inverse agonists, as well as full agonists of an opioid receptor. By opioid receptor agonist it is also meant to be inclusive of compounds that enhance the activity of opioid receptor agonist compounds produced within the body, as well as exogenous opioid receptor agonists (i.e., synthetic or naturally-occurring). Preferred opioid receptor agonists used in the present invention are partial or full agonists of the mu, delta, and/or kappa opioid receptors. Preferred opioid receptor agonists also include compounds from the opioid class of drugs, and more preferably opioids which act as analgesics. Examples of opioid receptor agonists useful in the present invention include, but are in no way limited to morphine, oxycodone, oxymorphone, hydromorphone, mepridine, methadone, fentanyl, sufentanil, alfentanil, remifentanil, carfentanil, lofentanil, codeine, hydrocodone, levorphanol, tramadol, D-Pen2, D-Pen5-enkephalin (DPDPE), U50, 488H (trans-3,4-dichloro-N-methyl-N-[2-pyrrolindinyl]-cyclohexanyl)-benzeneacetamide, endorphins, dynorphins, enkephalins, diamorphine (heroin), dihydrocodeine, nicomorphine, levomethadyl acetate hydrochloride (LAAM), ketobemidone, propoxyphene, dextropropoxyphene, dextromoramide, bezitramide, piritramide, pentazocine, phenazocine, buprenorphine, butorphanol, nalbufine or nalbuphine, dezocine, etorphine, tilidine, loperamide, diphenoxylate, paregoric and nalorphine.

Compositions of the present invention as well as methods described herein for their use may comprise more than one opioid receptor agonist and/or more than one alpha-2 receptor antagonist, formulated and/or administered in various combinations.

Preferred combinations of opioid receptor agonists and alpha-2 receptor antagonists used in the present invention include morphine and atipemazole, yohimbine, mirtazapine, or idazoxan, and oxycodone and atipemazole, yohimbine, mirtazapine, or idazoxan.

The dose of opioid receptor agonist included in the compositions of the present invention and used in the methodologies described herein is an amount that achieves an effective concentration and/or produces a desired therapeutic effect. For example, such a dosage may be an amount of opioid receptor agonist well known to the skilled artisan as having a therapeutic action or effect in a subject. Dosages of opioid receptor agonist producing, for example, an analgesic effect can typically range between about 0.02 mg/kg to 100 mg/kg, depending upon, but not limited to, the opioid receptor agonist selected, the route of administration, the frequency of administration, the formulation administered, and/or the condition being treated. Further, as demonstrated herein, co-administration of an opioid receptor agonist with an ultra-low dose of an alpha-2 receptor antagonist potentiates the analgesic effect of the opioid receptor agonist. Thus, when co-administered with an alpha-2 receptor antagonist, the amount or dose of opioid receptor agonist effective at producing a therapeutic effect may be lower than when the opioid receptor agonist is administered alone.

For purposes of the present invention, by “therapeutic effect” or “therapeutic activity” or “therapeutic action” it is meant a desired pharmacological activity of an opioid receptor agonist useful in the inhibition, reduction, prevention or treatment of a condition routinely treated with an opioid receptor agonist. Examples include, but are not limited to, pain, coughs, diarrhea, pulmonary edema and addiction to opioid receptor agonists. By these terms it is meant to include a pharmacological activity measurable as an end result, i.e. alleviation of pain or cough suppression, as well as a pharmacological activity associated with a mechanism of action linked to the end desired result. In a preferred embodiment, the “therapeutic effect” or “therapeutic activity” or “therapeutic action” is alleviation or management of pain.

For purposes of the present invention, by “potentiate”, it is meant that administration of the alpha-2 receptor antagonist enhances, extends or increases, at least partially, the therapeutic activity of an opioid receptor agonist and/or results in a decreased amount of opioid receptor agonist being required to produce a desired therapeutic effect. Thus, as will be understood by the skilled artisan upon reading this disclosure, the amount of opioid receptor agonist included in the combination therapy of the present invention may be decreased as compared to an established amount of the opioid receptor agonist when administered alone. The amount of the decrease for other opioid receptor agonists can be determined routinely by the skilled artisan based upon ratios described herein for morphine and atipemazole, morphine and yohimbine, morphine and mirtazapine, and/or morphine and idazoxan. By potentiate it is also meant to include any enhancement, extension or increase in therapeutic activity of an endogenous opioid receptor agonist in a subject upon administration of an ultra-low dose of an alpha-2 receptor antagonist.

This decrease in required amount of opioid receptor agonist to achieve the same or similar therapeutic benefit may decrease any unwanted side effects associated with opioid receptor agonist therapy. Thus, the combination therapies of the present invention also provide a means for decreasing unwanted side effects of opioid receptor agonist therapy alone.

By “antagonize” as used herein, it is meant an inhibition or decrease in therapeutic effect or action of an opioid receptor agonist resulting from addition of an alpha-2 receptor antagonist which renders the opioid receptor agonist ineffective or less effective therapeutically for the condition being treated.

By “tolerance” as used herein, it is meant a loss of level of drug-induced response and drug potency and is produced by many opioid receptor agonists, and particularly opioids. Chronic or acute tolerance can be a limiting factor in the clinical management of opioid drugs as opioid potency is decreased upon exposure to the opioid. By “chronic tolerance” it is meant a decrease in level of drug-induced response and drug potency which can develop after drug exposure over several or more days. “Acute tolerance” is a loss in drug potency which can develop after drug exposure over several hours (Fairbanks and Wilcox J. Pharmacol. Exp. Therapeutics. 1997 282:1408-1417; Kissin et al. Anesthesiology 1991 74:166-171). Loss of opioid drug potency may also be seen in pain conditions such as neuropathic pain without prior opioid drug exposure as neurobiological mechanisms underlying the genesis of tolerance and neuropathic pain are similar (Mao et al. Pain 1995 61:353-364). This is also referred to as acute tolerance. Tolerance has been explained in terms of opioid receptor desensitization or internalization although exposure to morphine, unlike most other mu opioid receptor agonists, does not produce receptor internalization. It has also been explained on the basis of an adaptive increase in levels of pain transmitters such as glutamic substance P or CGRP. Inhibition of tolerance and maintenance of opioid potency are important therapeutic goals in pain management which, as demonstrated herein, are achieved via the combination therapies of the present invention.

One skilled in the art would know which combination therapies would work to potentiate a therapeutic action of an opioid receptor agonist and/or inhibit acute or chronic opioid receptor agonist tolerance upon co-administration of an ultra-low dose of an alpha-2 receptor antagonist based upon the disclosure provided herein. For example, any given combination of opioid receptor agonist and alpha 2 receptor antagonist may be tested in animals using one or more available tests, including, but not limited to, tests for analgesia such as thermal, mechanical and the like, or any other tests useful for assessing antinociception as well as other therapeutic actions of opioid receptor agonists. Non-limiting examples for testing analgesia include the thermal rat tail flick and mechanical rat paw pressure antinociception assays.

The ability of exemplary combination therapies of the present invention to potentiate the analgesic action of an opioid receptor agonist and/or inhibit acute or chronic opioid receptor agonist tolerance upon co-administration of an ultra-low dose of an alpha-2 receptor antagonist was demonstrated in tests of both thermal (rat tail flick) and mechanical (rat paw pressure) antinociception. In these experiments, the opioid receptor agonist was the opioid morphine. The alpha-2 receptor antagonists included atipemazole, yohimbine, mirtazapine and idazoxan.

Initial studies showed that atipemazole administered intrathecally antagonized the analgesic action of the alpha-2 receptor agonist clonidine at doses greater than 1 microgram. FIGS. 1A and 1B show the effects of atipemazole on the clonidine-induced analgesia in the tail flick (FIG. 1A) and paw pressure test (FIG. 1B). Injection of clonidine (200 nmoles), an alpha-2 receptor agonist, produced a maximal analgesic response in the tail flick test and a lesser effect in the paw pressure test. Co-administration of three different doses of atipemazole produced a dose-related decrease in the peak clonidine analgesia in the tail flick test, the highest drug dose (10 μg) almost abolishing the response. Atipemazole also decreased clonidine response in the paw pressure test but only at the highest dose. These experiments established that the atipemazole could block clonidine analgesia, an effect consistent with its identity as an alpha-2 receptor antagonist.

Thus, for all subsequent tests involving atipemazole interactions with morphine, the atipemazole dose was lowered to the exemplary ultra-low doses of 0.08 ng and 0.8 ng, representing a 12,000-fold to 120,000-fold decrease in the dose producing maximal alpha-2 receptor blockade.

The effects of ultra-low doses of atipemazole on the development of acute tolerance to morphine were examined. The development of acute tolerance is indicated by a rapid decline of the analgesic effect following repeated administration of morphine over several hours. In these experiments, acute tolerance was produced by delivering three intrathecal successive injections of morphine (15 μg) at 90 minute intervals. In subsequent experiments, morphine was combined with a fixed dose of atipemazole (0.8 ng). The effect of atipemazole alone (0.8 ng) or normal saline (20 μl) was also evaluated by injecting these at 90 minute intervals. Pain responses were evaluated in the tail flick and paw pressure test at 30 minute intervals. Twenty-four hours after the drug treatment, cumulative dose-response curves (DRCs) for the action of morphine in each treatment group were obtained to establish the drug potency index. This index, represented by the morphine ED₅₀ or Ed₅₀ value, (effective dose in 50% of animals tested) was calculated from the cumulative dose-response curves. Tolerance was indicated by a rightward shift in the morphine dose-response curve and an increase in the morphine ED₅₀ value.

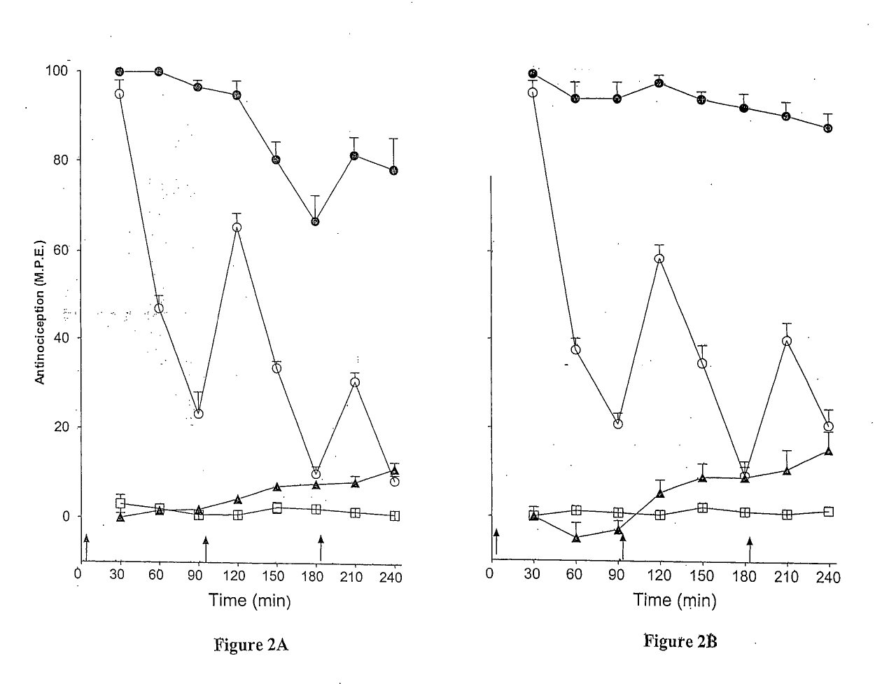

FIGS. 2A and 2B illustrate effects of an ultra-low dose of atipemazole on the acute tolerance to the analgesic actions of spinal morphine. Administration of 3 successive doses of morphine (15 μg) at 90 minute intervals resulted in a rapid and progressive reduction of the analgesic response. At the end of the 240 minute test period, the analgesic effect of morphine observed after the first injection had declined by nearly 80%. However, administration of atipemazole (0.8 ng) with morphine prevented the decline of the analgesic effect of morphine. Indeed, the response to the combination remained near maximal value during the entire test period. The repeated administration of atipemazole alone produced an incremental but weak analgesic response. The three successive saline injections did not produce significant analgesic effect in either test.

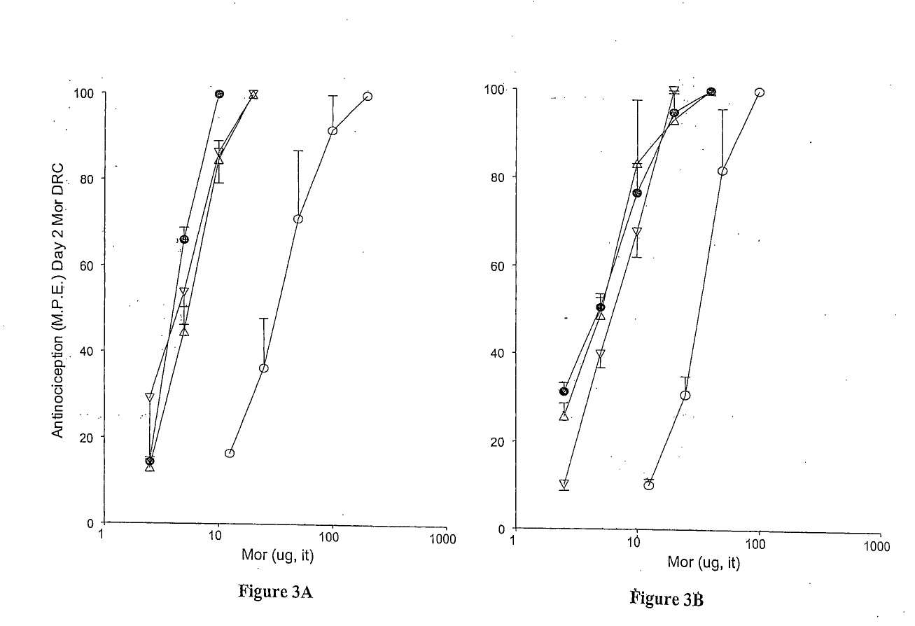

The cumulative dose-response curves for the acute analgesic action of morphine in the four treatment groups in FIGS. 2A and 2B, derived 24 hours after the first morphine injection, are shown in FIGS. 3A and 3B, respectively. Ascending doses of acute morphine produced dose-related analgesia in both the tail flick and paw pressure tests. In animals that had received repeated morphine injections, the cumulative dose-response curve was shifted to the right, reflecting a decline in the morphine potency. However, this shift did not occur in the group receiving a combination therapy of the present invention. Instead, the dose-response curve obtained in this group coincided with that derived in the saline or atipemazole (alone) group. Thus, co-administration of an ultra-low dose of an alpha-2 receptor antagonist prevented the rightward shift of the opioid dose-response curve that signifies the development of opioid tolerance.

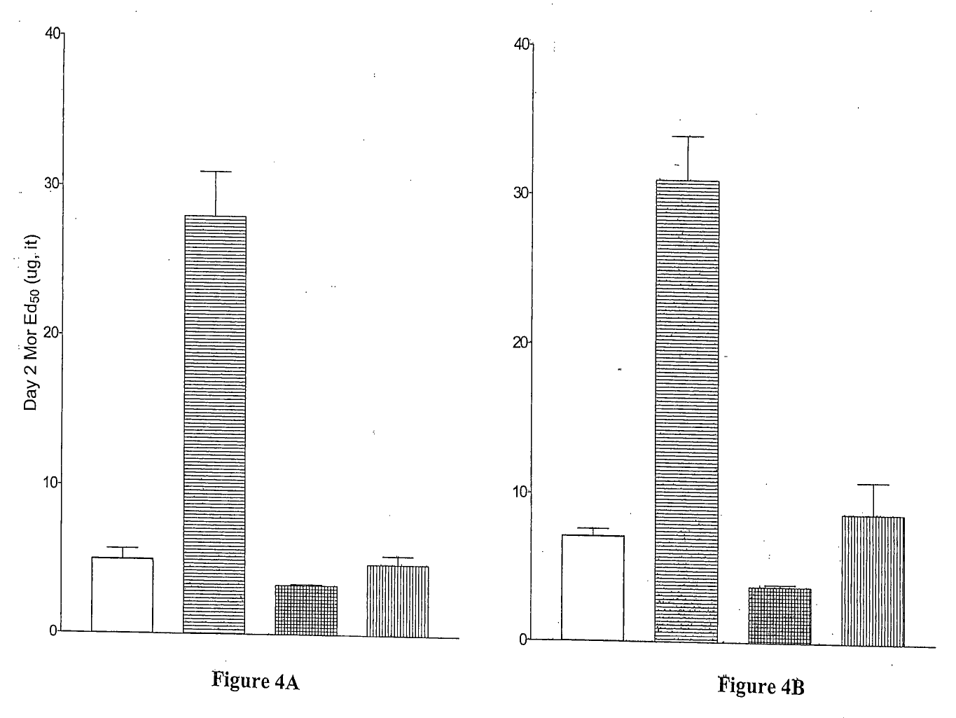

The ED₅₀ values, an index of drug potency, derived from the cumulative dose-response curves of FIGS. 3A and 3B are represented in FIGS. 4A and 4B, respectively. As shown therein, in the saline-treated control group, the ED₅₀ value of morphine approximated 5 and 8 μg in the tail flick and paw pressure test, respectively. The group receiving repeated morphine injections showed nearly a 5-fold increase in the tail flick and a 4-fold increase in the paw pressure test, reflecting a highly significant loss of morphine potency. Introduction of atipemazole with morphine, however, prevented the increase in ED₅₀ values in both tests. In fact, the ED₅₀ values in the atipemazole morphine combination group were not significantly different from those in the control saline group, indicating that morphine potency was completely maintained in the presence of the alpha-2 receptor antagonist atipemazole.

Thus, as shown by these experiments ultra-low dose administration of an alpha-2 receptor antagonist such as atipemazole very effectively inhibits the development of acute tolerance to an opioid such as morphine.

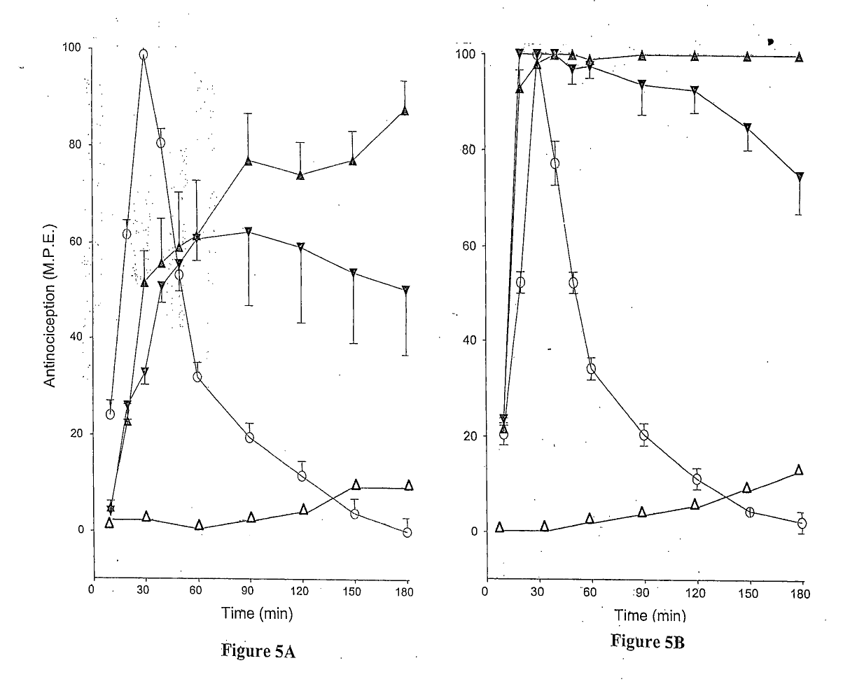

Further, as shown in FIGS. 5A and 5B, alpha-2 receptor antagonists such as atipemazole, when administered at an ultra-low dose of 0.8 or 0.08 ng, potentiate opioid analgesia. The fact that atipemazole exerts these effects when given intrathecally suggests that it exerts a direct action on spinal nociceptive neurons.

The analgesic effect of ultra-low dose atipemazole, when administered alone, depicted in FIGS. 2 and 5 may also be indicative of this therapy potentiating endogenous opioids such as endorphins (examples include beta-endorphins dynorphins and enkephalins) as well. Thus, the present invention also provides methods for potentiating the therapeutic actions of an endogenous opioid in a subject (not being administered an exogenous opioid) upon administration of an ultra-low dose alpha-2 receptor antagonist to the subject.

Similar effects were observed with the alpha-2 receptor antagonist yohimbine.

As shown in FIGS. 15A and 15B, yohimbine administered intrathecally antagonized the analgesic action of the alpha-2 receptor agonist clonidine at a 30 μg dose. FIGS. 15A and 15B show the effects of yohimbine on the clonidine-induced analgesia in the tail flick (FIG. 15A) and paw pressure test (FIG. 15B). Injection of clonidine (13.3 μg), an alpha-2 receptor agonist, produced a maximal analgesic response in the tail flick test and a lesser effect in the paw pressure test. Co-administration of yohimbine at 30 μg decreased significantly peak clonidine analgesia in the tail flick test. Yohimbine at 30 μg also almost abolished clonidine analgesia in the paw pressure test. These experiments established that the yohimbine, like atipemazole, blocks clonidine analgesia, an effect consistent with its identity as an alpha-2 receptor antagonist.

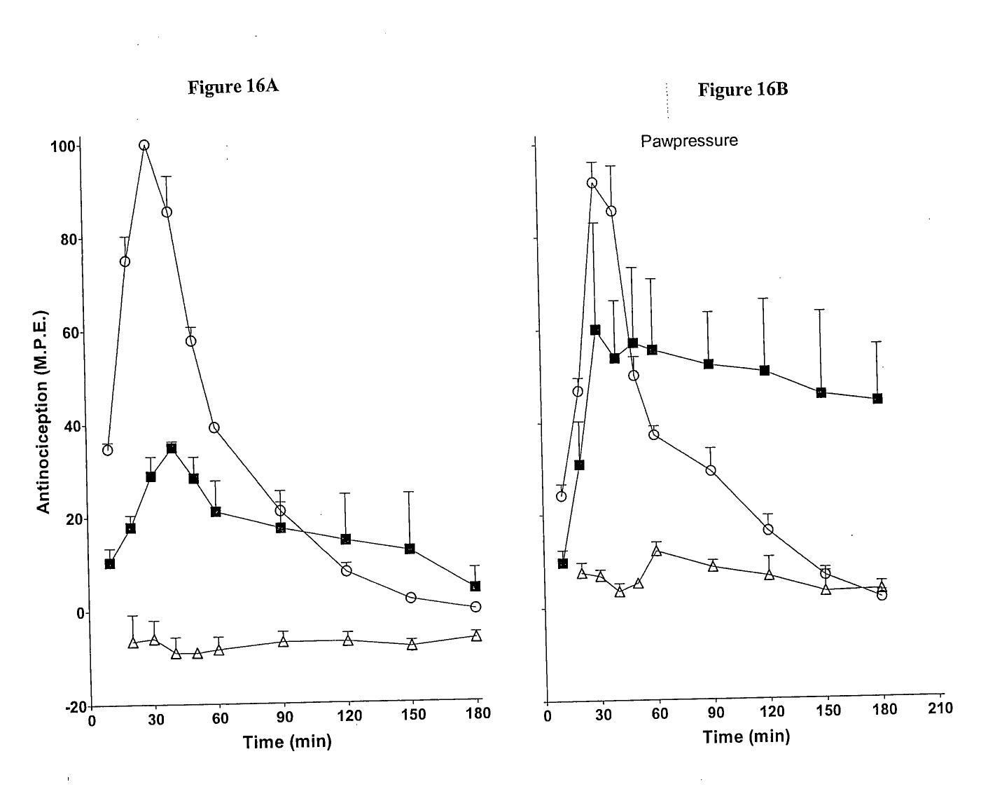

Similar inhibition of morphine analgesia was observed upon co-administration with yohimbine at 30 μg. See FIGS. 16A and 16B. In these experiments, yohimbine was less effective at inhibition of morphine analgesia a compared to inhibition of clonidine analgesia in the paw pressure test. See FIG. 16B versus FIG. 15B.

For all subsequent tests involving yohimbine interactions with morphine, the yohimbine dose was lowered to exemplary ultra-low doses of 0.0048 ng, 0.024 ng, 0.24 ng, 2.4 ng and 5 ng, representing a 6,000-fold to 6,250,000-fold decrease in the dose producing maximal alpha-2 receptor blockade.

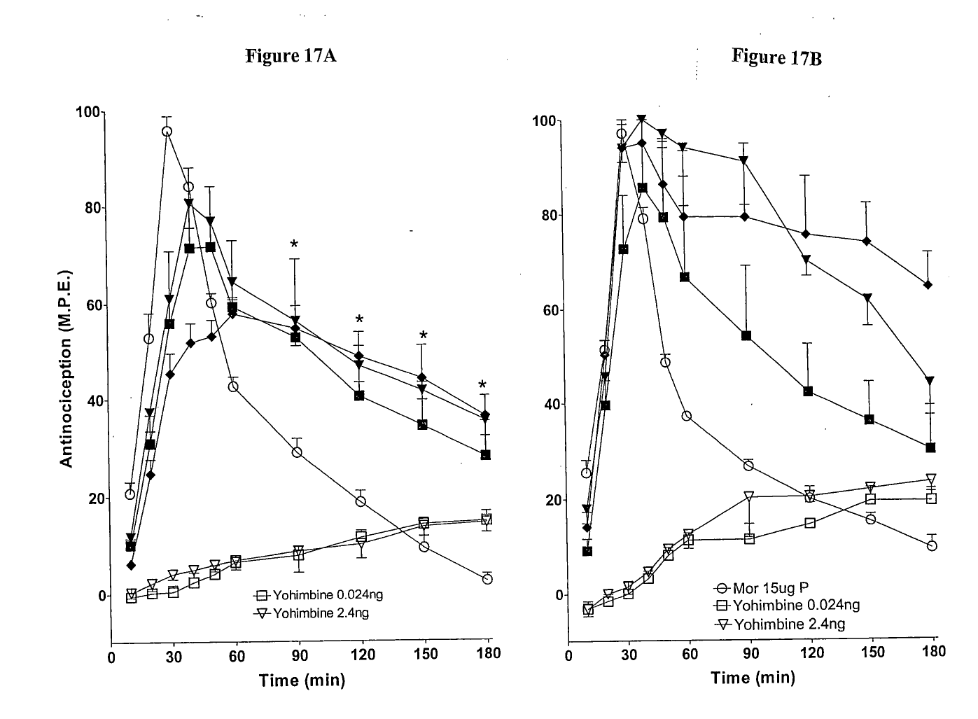

As shown in FIG. 17A and FIG. 17B, administration of a single dose of morphine (15 μg) produced analgesia in the rat tail flick test (FIG. 17A) and rat paw pressure test (FIG. 17B) that peaked at 30 minutes and terminated at 120 minutes. Addition of ultra-low doses of yohimbine (0.24, 2.4 and 5 ng) extended morphine analgesia in the rat tail flick test and augmented and extended the response to morphine in the rat paw pressure test. This profile of yohimbine ultra-low dose is similar to atipemazole ultra-low dose discussed supra.

The effects of ultra-low doses of yohimbine on the development of acute tolerance to morphine were also examined. In similar fashion to experiments with atipemazole, acute tolerance was produced by delivering three intrathecal successive injections of morphine (15 μg) at 90 minute intervals. In subsequent experiments, morphine was combined with fixed doses of yohimbine at 0.0048, 0.024, and 0.24 ng. The effect of yohimbine alone (0.024 ng) or normal saline (20 μl) was also evaluated by injecting these at 90 minute intervals. Pain responses were evaluated in the tail flick and paw pressure test at 30 minute intervals. Twenty-four hours after the drug treatment, cumulative dose-response curves (DRCs) for the action of morphine in each treatment group were obtained to establish the drug potency index. This index, represented by the morphine ED₅₀ or Ed₅₀ value, (effective dose in 50% of animals tested) was calculated from the cumulative dose-response curves. Tolerance was indicated by a rightward shift in the morphine dose-response curve and an increase in the morphine ED₅₀ value.

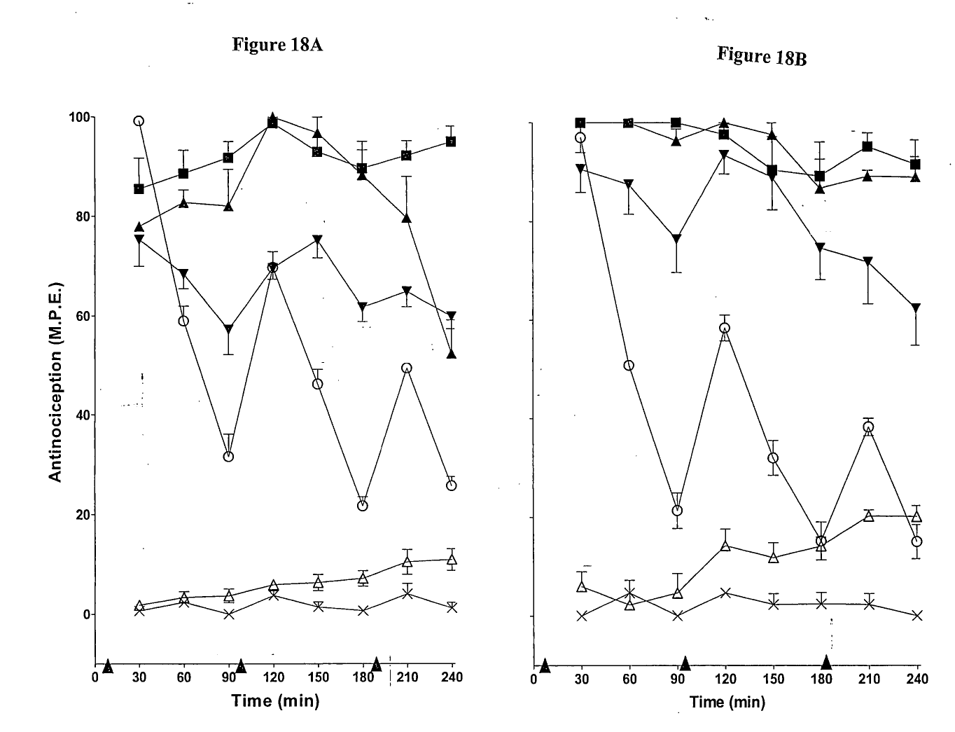

FIGS. 18A and 18B illustrate effects of an ultra-low dose of yohimbine on the acute tolerance to the analgesic actions of spinal morphine. Administration of 3 successive doses of morphine (15 μg) at 90 minute intervals resulted in a rapid and progressive reduction of the analgesic response. At the end of the 240 minute test period, the analgesic effect of morphine observed after the first injection had declined by nearly 80%. However, administration of morphine with yohimbine at a dose of either 0.0048 ng, 0.024 ng or 0.24 ng prevented the decline of the analgesic effect of morphine. Indeed, the response to the combination remained near maximal value, particularly in animals administered either 0.0048 ng or 0.024 ng yohimbine during the entire test period. Repeated administration of yohimbine (0.024 ng) alone or saline produced no significant analgesic response.

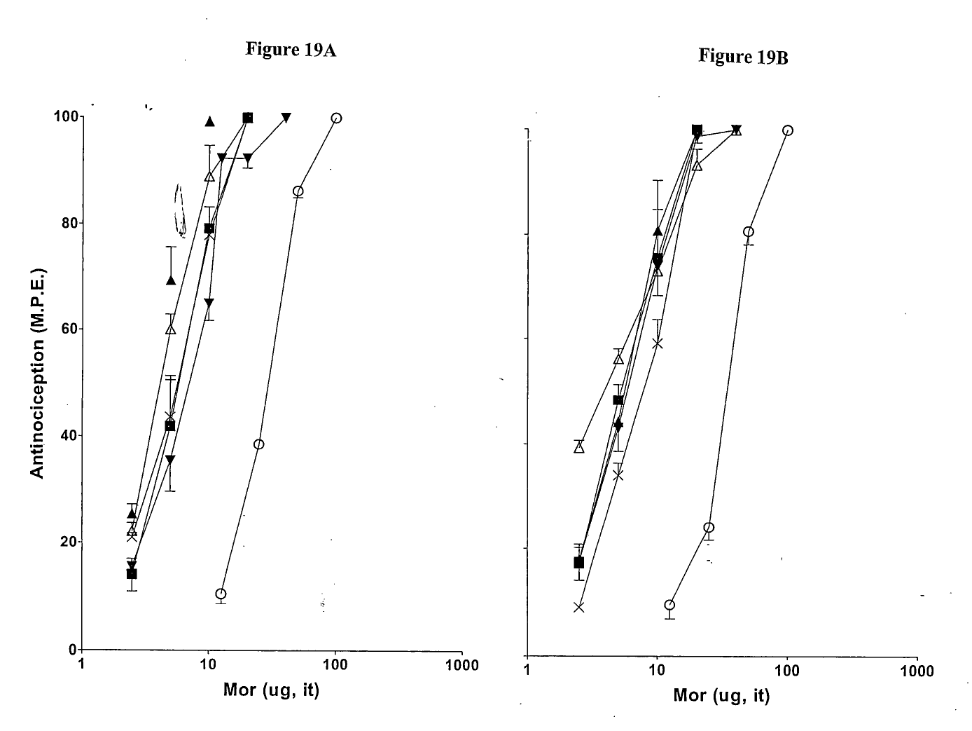

The cumulative dose-response curves for the acute analgesic action of morphine in the six treatment groups in FIGS. 18A and 18B, derived 24 hours after the first morphine injection, are shown in FIGS. 19A and 19B, respectively. Ascending doses of acute morphine produced dose-related analgesia in both the tail flick and paw pressure tests. In animals that had received repeated morphine injections, the cumulative dose-response curve was shifted to the right, reflecting a decline in the morphine potency. However, this shift did not occur in the group receiving a combination therapy of the present invention. Instead, the dose-response curve obtained in this group coincided with that derived in the saline or yohimbine (alone) group. Thus, like atipemazole, co-administration of an ultra-low dose of a second alpha-2 receptor antagonist, yohimbine, also prevented the rightward shift of the opioid receptor agonist dose-response curve, a response that signifies the development of opioid receptor agonist tolerance.

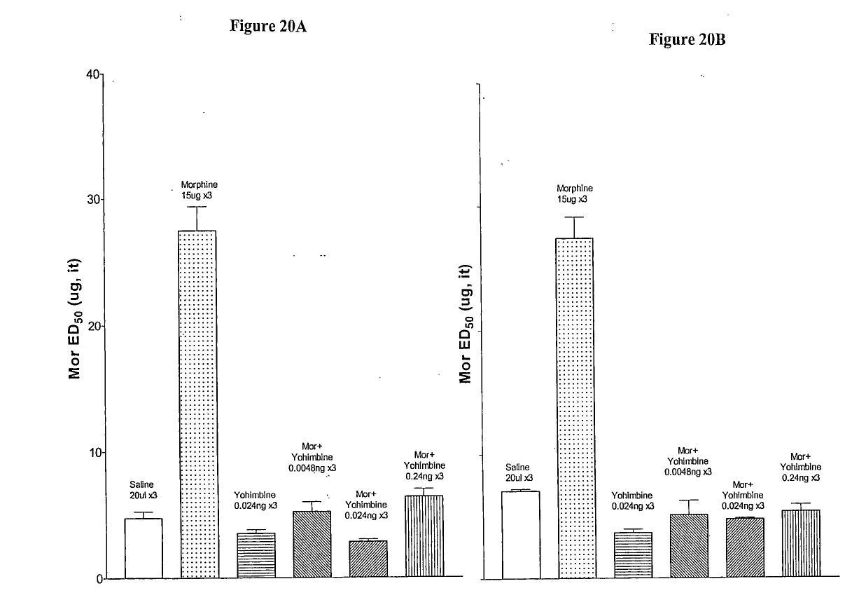

The ED₅₀ values, an index of drug potency, derived from the cumulative dose-response curves of FIGS. 19A and 19B are represented in FIGS. 20A and 20B, respectively. As shown therein, in the saline-treated control group, the ED₅₀ value of morphine approximated 5 and 7 μg in the tail flick and paw pressure test, respectively. The group receiving repeated morphine injections showed nearly a 5-fold increase in the tail flick and a 4-fold increase in the paw pressure test, reflecting a highly significant loss of morphine potency. Introduction of yohimbine with morphine, however, prevented the increase in ED₅₀ values in both tests. In fact, the ED₅₀ values in the yohimbine-morphine combination group were either lower or not significantly different from those in the control saline group, indicating that morphine potency was also completely maintained in the presence of this second alpha-2 receptor antagonist yohimbine.

Similar effects were observed with the alpha-2 receptor antagonist idazoxan.

As shown in FIGS. 28A and 28B, idazoxan administered intrathecally antagonized the analgesic action of the alpha-2 receptor agonist clonidine at a 10 μg dose. FIGS. 28A and 28B show the effects of idazoxan on the clonidine-induced analgesia in the tail flick (FIG. 28A) and paw pressure test (FIG. 28B). Injection of clonidine (13.3 μg), an alpha-2 receptor agonist, produced a maximal analgesic response in the tail flick test and a lesser effect in the paw pressure test. Co-administration of idazoxan at 10 μg decreased significantly peak clonidine analgesia in the tail flick test. Mirtazapine at 10 μg also almost abolished clonidine analgesia in the paw pressure test. These experiments established that idazoxan, like yohimbine and atipemazole, blocks clonidine analgesia, an effect consistent with its identity as an alpha-2 receptor antagonist.

For all subsequent tests involving idazoxan interactions with morphine, the idazoxan doses were lowered to the exemplary ultra-low doses of 0.008 ng, 0.016 ng and 0.08 ng, representing a 125,000-fold to 1,250,000-fold decrease in the dose producing maximal alpha-2 receptor blockade.

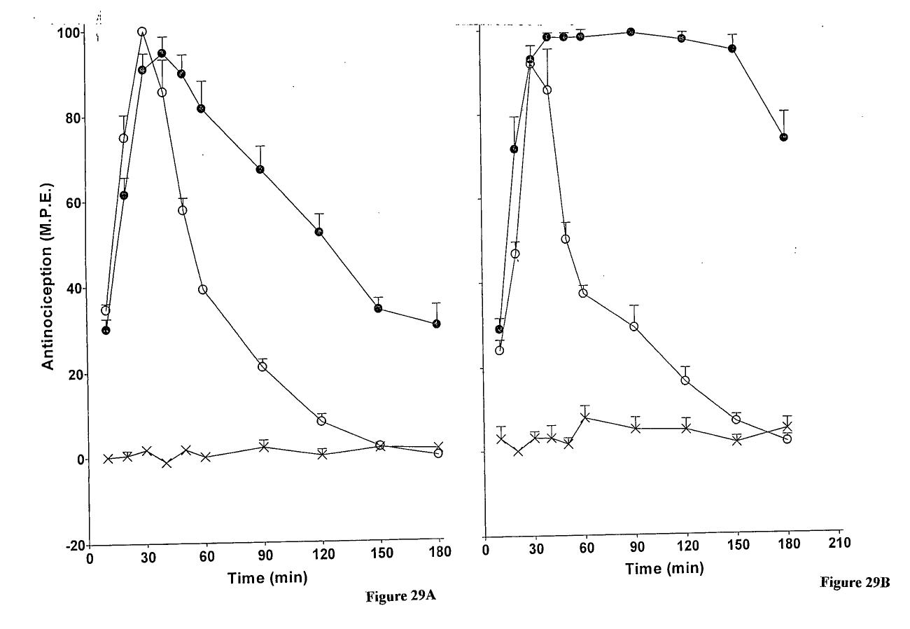

As shown in FIG. 29A and FIG. 29B, administration of a single dose of morphine (15 μg) produced analgesia in the rat tail flick test (FIG. 29A) and rat paw pressure test (FIG. 29B) that peaked at 30 minutes and terminated at approximately 120 minutes. Addition of an ultra-low dose of idazoxan (0.08 ng) significantly extended morphine analgesia in both the rat tail flick test (FIG. 29A) and the rat paw pressure test (FIG. 29B). Further, administration of 0.08 ng idazoxan augmented peak morphine analgesia in the rat par pressure test.

The effects of ultra-low doses of idazoxan on the development of acute tolerance to morphine were also examined. In similar fashion to experiments with atipemazole and yohimbine, acute tolerance was produced by delivering three intrathecal successive injections of morphine (15 μg) at 90 minute intervals. In subsequent experiments, morphine was combined with fixed doses of idazoxan at 0.008, 0.016 and 0.08 ng. The effects of normal saline (20 μl) and idazoxan alone at 0.008 and 0.016 ng were also evaluated by injection at 90 minute intervals. Pain responses were evaluated in the tail flick and paw pressure test at 30 minute intervals. Twenty-four hours after the drug treatment, cumulative dose-response curves (DRCs) for the action of morphine in each treatment group were obtained to establish the drug potency index. This index, represented by the morphine ED₅₀ or Ed₅₀ value, (effective dose in 50% of animals tested) was calculated from the cumulative dose-response curves. Tolerance was indicated by a rightward shift in the morphine dose-response curve and an increase in the morphine ED₅₀ value.

FIGS. 29A and 29B illustrate effects of an ultra-low dose of idazoxan on the acute tolerance to the analgesic actions of spinal morphine. Administration of 3 successive doses of morphine (15 μg) at 90 minute intervals resulted in a rapid and progressive reduction of the analgesic response. At the end of the 240 minute test period, the analgesic effect of morphine observed after the first injection had declined by nearly 80%. However, administration of morphine with ultra-low doses of idazoxan arrested the decline of the analgesic effect of morphine analgesia in the paw pressure test, maintaining analgesia near peak levels. Co-injection with the same ultra-low doses of idazoxan in the tail flick test were less effective at arresting the decline of the analgesic effect and the lowest dose of 0.008 ng reduced the peak morphine analgesia. Repeated administration of idazoxan or saline produced no significant analgesic response.

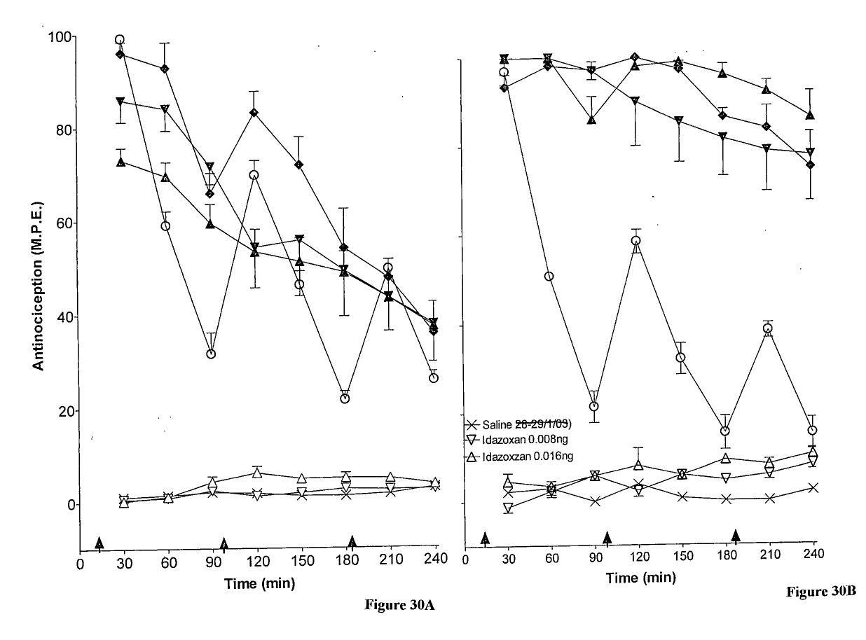

The cumulative dose-response curves for the acute analgesic action of morphine in the seven treatment groups in FIGS. 29A and 29B, derived 24 hours after the first morphine injection, are shown in FIGS. 30A and 30B, respectively. Repeated morphine treatment resulted in a parallel right shift of the morphine dose response curve relative to the saline treatment. Idazoxan, at ultra-low doses of 0.008 ng, 0.016 ng and 0.008 ng, prevented the rightward shift in both the tail flick test (FIG. 30A) and the paw pressure test (FIG. 30B). Thus, co-administration of an ultra-low dose of a third alpha-2 receptor antagonist, idazoxan, also prevented the rightward shift of the opioid receptor agonist dose-response curve, a response that signifies the development of opioid receptor agonist tolerance.

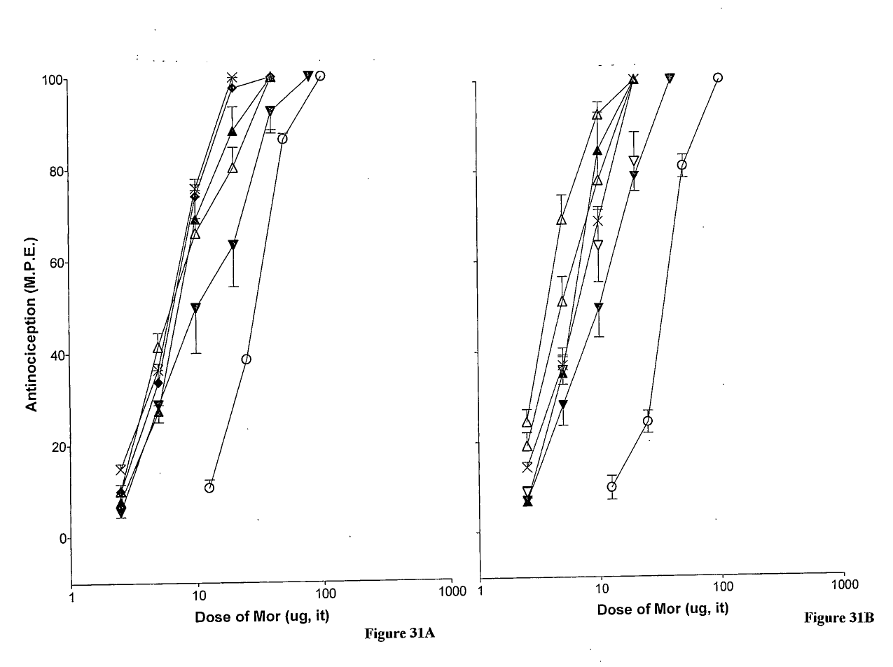

The ED₅₀ values, an index of drug potency, derived from the cumulative dose-response curves of FIGS. 30A and 30B are represented in FIGS. 31A and 31B, respectively. As shown therein, ultra-low dose idazoxan (0.008, 0.016 and 0.08 ng) co-injection prevented the increase in ED50 in both the tail flick test and the paw pressure test. Thus, morphine potency was also maintained in the presence of this third alpha-2 receptor antagonist mirtazapine.

Similar effects were observed with the alpha-2 receptor antagonist mirtazapine, particularly in the paw pressure test.

As shown in FIGS. 21A and 21B, mirtazapine administered intrathecally antagonized the analgesic action of the alpha-2 receptor agonist clonidine at a 2 μg dose. FIGS. 21A and 21B show the effects of mirtazapine on the clonidine-induced analgesia in the tail flick (FIG. 21A) and paw pressure test (FIG. 21B). Injection of clonidine (13.3 μg), an alpha-2 receptor agonist, produced a maximal analgesic response in the tail flick test and a lesser effect in the paw pressure test. Co-administration of mirtazapine at 2 μg decreased significantly peak clonidine analgesia in the tail flick test. Mirtazapine at 2 μg also almost abolished clonidine analgesia in the paw pressure test. These experiments established that mirtazapine, like yohimbine and atipemazole, blocks clonidine analgesia, an effect consistent with its identity as an alpha-2 receptor antagonist.

For all subsequent tests involving mirtazapine interactions with morphine, the mirtazapine dose was lowered to exemplary ultra-low doses of 0.02 ng and 0.2 ng, representing a 1,000-fold to 10,000-fold decrease in the dose producing maximal alpha-2 receptor blockade.

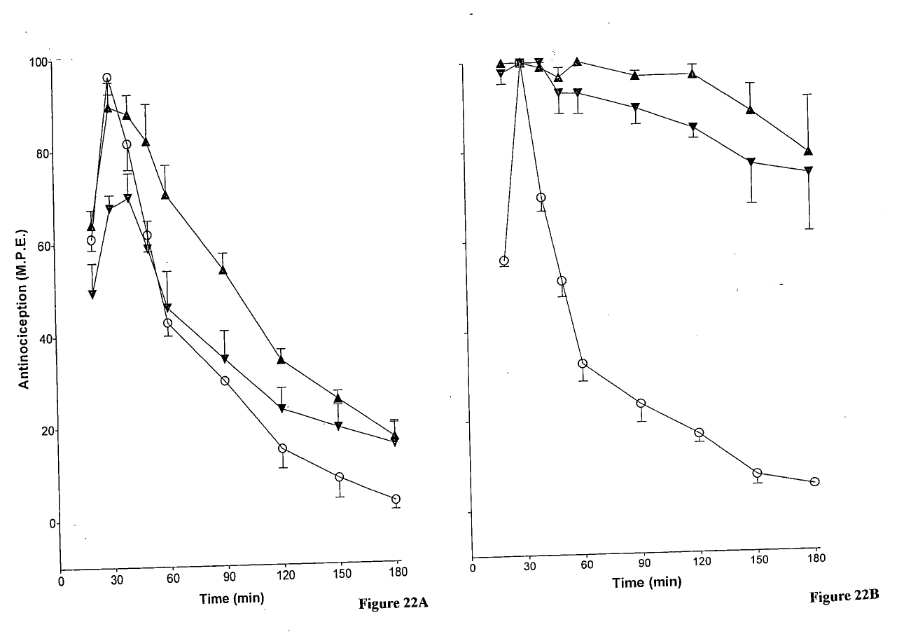

As shown in FIG. 22A and FIG. 22B, administration of a single dose of morphine (15 μg) produced analgesia in the rat tail flick test (FIG. 22A) and rat paw pressure test (FIG. 22B) that peaked at 30 minutes and terminated at approximately 120 minutes. Addition of ultra-low doses of mirtazapine (0.02 and 0.2 ng) significantly extended morphine analgesia in the rat paw pressure test (FIG. 22B). Further, while administration of 0.2 ng mirtazapine reduced the peak morphine analgesia, mirtazapine at 0.02 and 0.2 ng extended morphine analgesia in the rat tail flick test, particularly at the lower dose of 0.02 ng.

The effects of ultra-low doses of mirtazapine on the development of acute tolerance to morphine were also examined. In similar fashion to experiments with atipemazole and yohimbine, acute tolerance was produced by delivering three intrathecal successive injections of morphine (15 μg) at 90 minute intervals. In subsequent experiments, morphine was combined with fixed doses of mirtazapine at 0.02 and 0.2 ng. The effect of normal saline (20 μl) was also evaluated by injection at 90 minute intervals. Pain responses were evaluated in the tail flick and paw pressure test at 30 minute intervals. Twenty-four hours after the drug treatment, cumulative dose-response curves (DRCs) for the action of morphine in each treatment group were obtained to establish the drug potency index. This index, represented by the morphine ED₅₀ or Ed₅₀ value, (effective dose in 50% of animals tested) was calculated from the cumulative dose-response curves. Tolerance was indicated by a rightward shift in the morphine dose-response curve and an increase in the morphine ED₅₀ value.

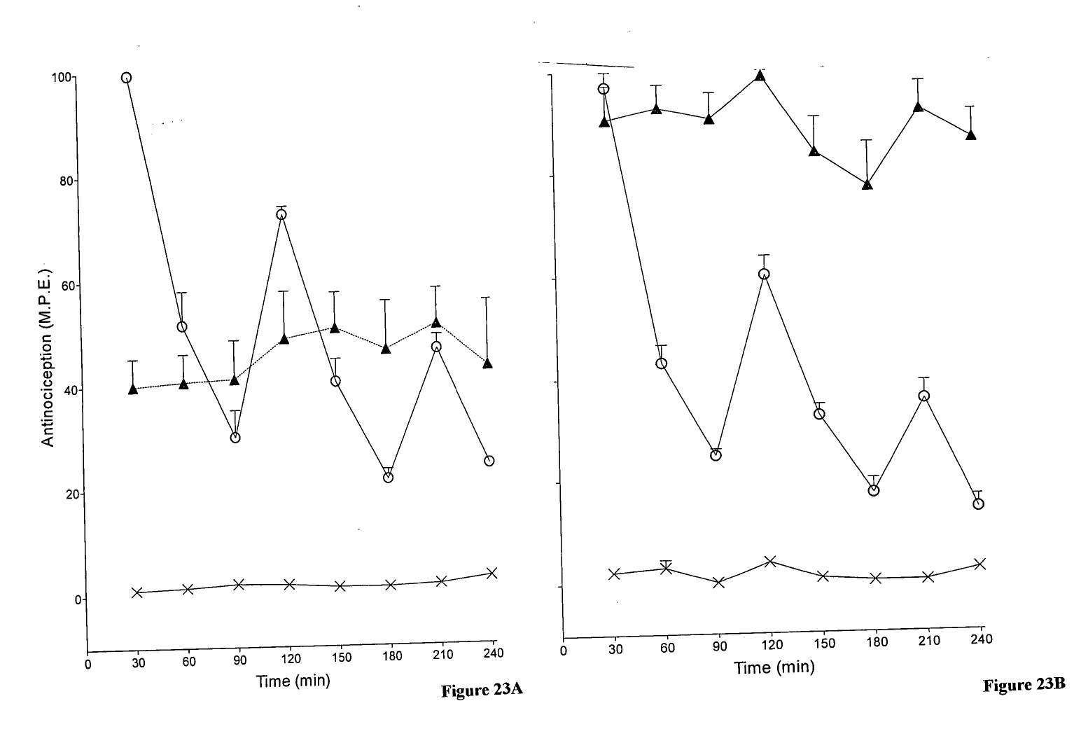

FIGS. 23A and 23B illustrate effects of an ultra-low dose of mirtazapine on the acute tolerance to the analgesic actions of spinal morphine. Administration of 3 successive doses of morphine (15 μg) at 90 minute intervals resulted in a rapid and progressive reduction of the analgesic response. At the end of the 240 minute test period, the analgesic effect of morphine observed after the first injection had declined by nearly 80%. However, administration of morphine with mirtazapine at a dose of 0.02 ng arrested the decline of the analgesic effect of morphine analgesia in the paw pressure test, maintaining analgesia near peak levels. Co-injection with this same ultra-low dose of mirtazapine in the tail flick test was less effective at arresting the decline of the analgesic effect and again reduced the peak morphine analgesia. Repeated administration of saline produced no significant analgesic response.

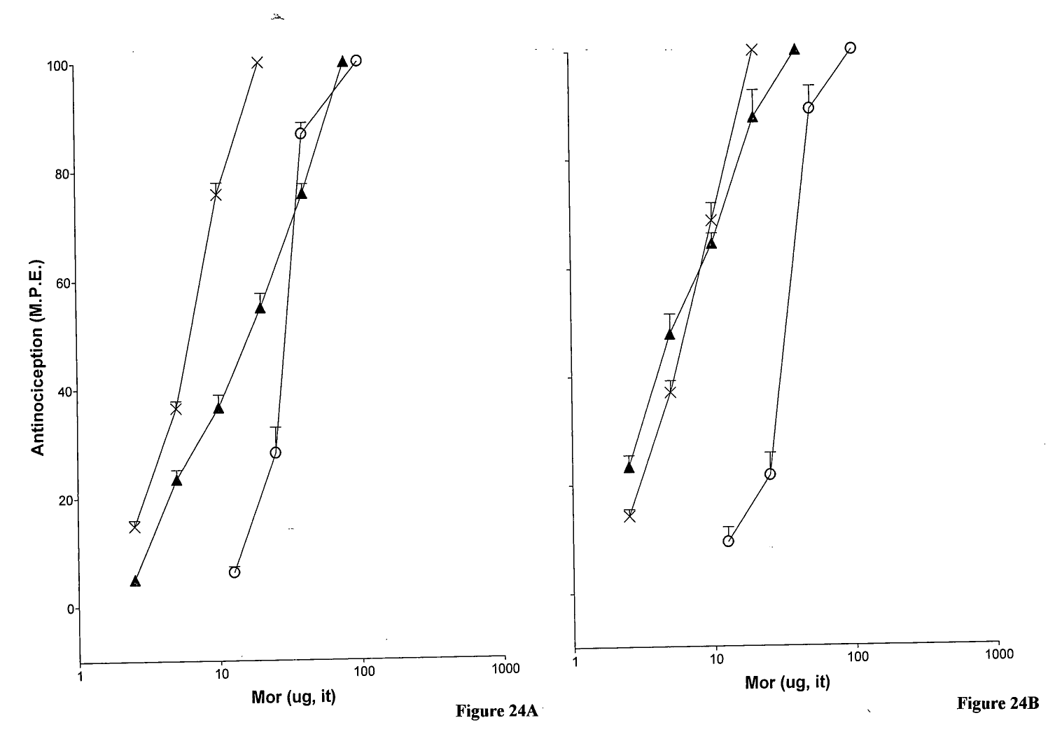

The cumulative dose-response curves for the acute analgesic action of morphine in the three treatment groups in FIGS. 23A and 23B, derived 24 hours after the first morphine injection, are shown in FIGS. 24A and 24B, respectively. Repeated morphine treatment resulted in a parallel right shift of the morphine does response curve relative to the saline treatment. Mirtazapine, at an ultra-low dose, prevented the rightward shift in the paw pressure test. In the tail flick test, however, the curves obtained in the morphine and morphine-mirtazapine groups showed an overlap at upper dose range of the opioid agonist. Thus, co-administration of an ultra-low dose of a third alpha-2 receptor antagonist, mirtazapine, at least in the paw pressure test, also prevented the rightward shift of the opioid receptor agonist dose-response curve, a response that signifies the development of opioid receptor agonist tolerance.

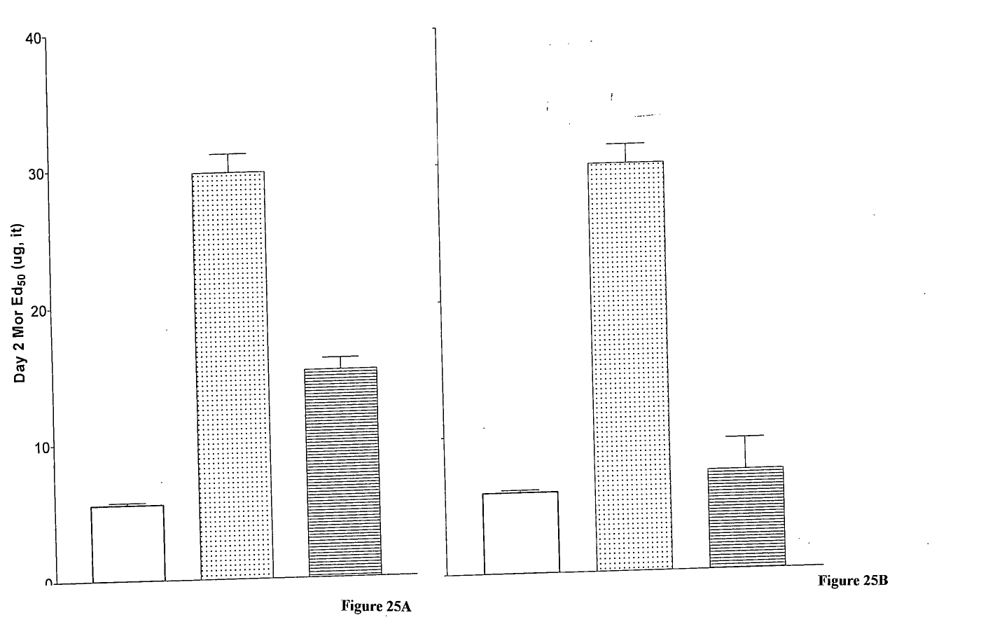

The ED₅₀ values, an index of drug potency, derived from the cumulative dose-response curves of FIGS. 24A and 24B are represented in FIGS. 25A and 25B, respectively. As shown therein, ultra-low dose mirtazapine (0.02 ng) co-injection completely prevented the increase in ED50 in the paw pressure test and partially prevented the increase in ED50 in the tail flick test. Thus, morphine potency was also maintained in the presence of this third alpha-2 receptor antagonist mirtazapine.

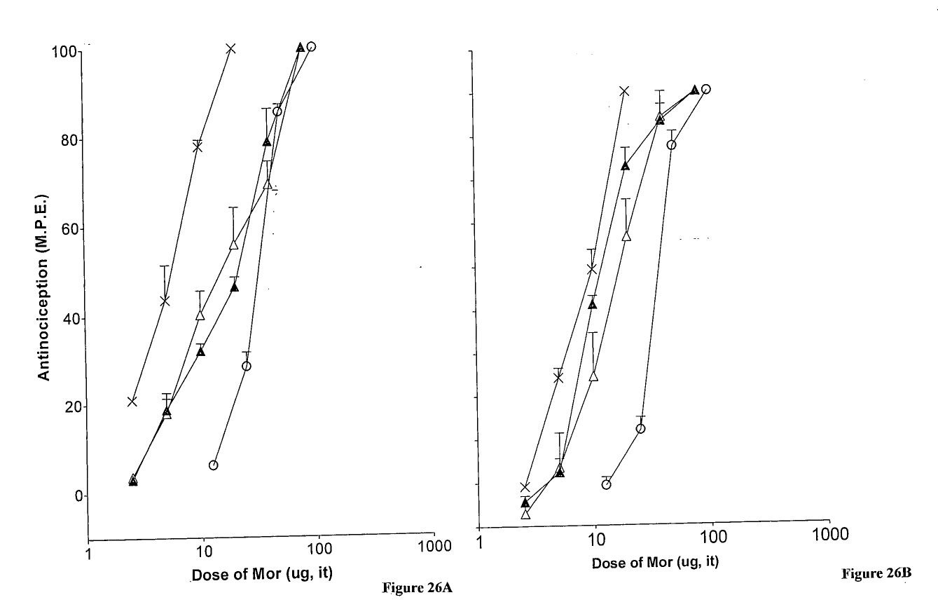

The effects of pretreatment with a single ultra-low dose of mirtazapine on the loss of analgesia produced by repeated morphine injections were also examined. In these experiments, an intrathecal mirtazapine dose was delivered 30 minutes prior to three successive injections of morphine or saline. FIGS. 26A and 26B show the cumulative morphine dose-response curves obtained 24 hours after treatment. Like the experiments depicted in FIGS. 23A and 23B and 24A and B, pretreatment with an ultra-low dose or mirtazapine was more effective in the paw pressure test at preventing the right shift of the dose response curve resulting from repeated opioid injection.

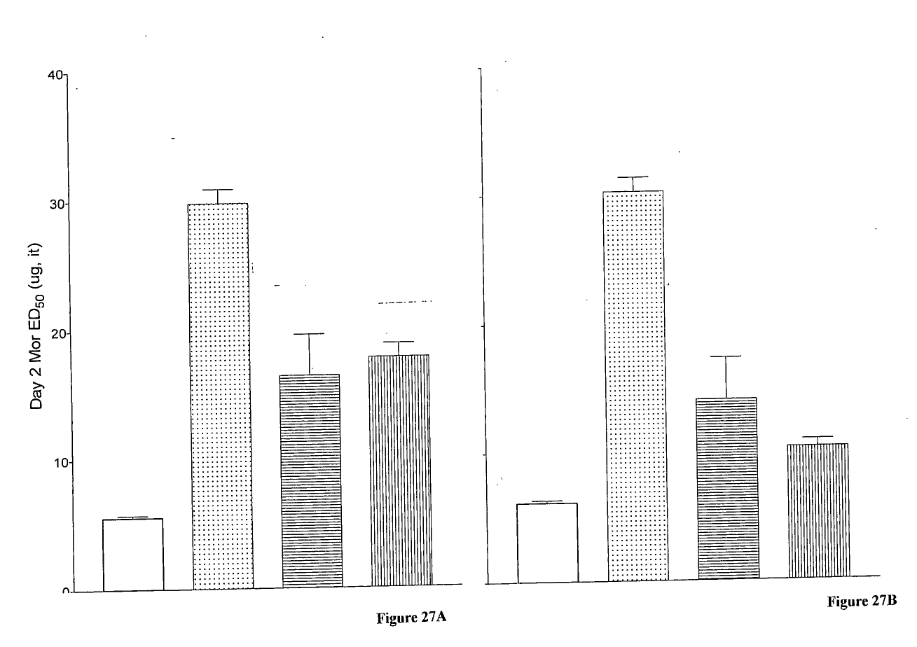

The morphine ED₅₀ values, reflecting potency of morphine derived from the dose response curves depicted in FIGS. 26A and 26B are depicted in FIGS. 27A and 27B. As shown therein, in both the tail flick test (FIG. 27A) and the paw pressure test (FIG. 27B), repeated morphine treatment produced a 3 to 4 fold increase in the ED50 values over those produced by repeated saline treatment, reflecting a loss of drug potency. Single mirtazapine exposure, 30 minutes prior to repeated morphine, partially prevented the increase in ED50 in the tail flick test and completely prevented the increase in ED50 in the paw pressure test. Thus, ultra-low dose mirtazapine pre-exposure inhibited loss of potency induced by repeated opioid treatment.

Thus, as shown by these experiments, ultra-low dose administration of alpha-2 receptor antagonists such as atipemazole, yohimbine, mirtazapine and idazoxan very effectively inhibit the development of acute tolerance to an opioid receptor agonist such as morphine.

Further, as shown in FIGS. 5A and 5B, alpha-2 receptor antagonists such as atipemazole, when administered at an ultra-low dose such as 0.8 or 0.08 ng, potentiate opioid receptor agonist analgesia. The fact that atipemazole exerts these effects when given intrathecally suggests that it exerts a direct action on spinal nociceptive neurons.

The effects of ultra-low doses of atipemazole on the development of chronic tolerance to morphine were also examined. The development of acute tolerance is indicated by a rapid decline of morphine effect following administration of daily doses of morphine over several days. In these experiments, animals were given a single intrathecal injection of morphine (15 μg) daily between 9 AM and 11 AM for 5 days. Nociceptive testing was performed once before drug treatment to establish the control response level, and 30 minutes after drug administration to determine the drug effect. Peak antinociceptive response to morphine occurs 30 minutes post-injection. On day 6, cumulative morphine dose-response curves were generated to determine acute opioid receptor agonist potency in the control and treatment groups. Each animal was given ascending doses of morphine at 30 minute intervals and tested 25 minutes after each injection. This protocol was continued until a maximal antinociceptive response was obtained in both the tail flick and paw pressure test. The morphine dose-response curves were constructed and the ED₅₀ values of morphine were determined from each curve. The development of a morphine-tolerant state was revealed by a progressive decline in the daily antinociceptive effect of morphine over the 5-day treatment period, a rightward shift in the acute morphine dose-response curve, and a significant increase in the morphine ED₅₀ value.

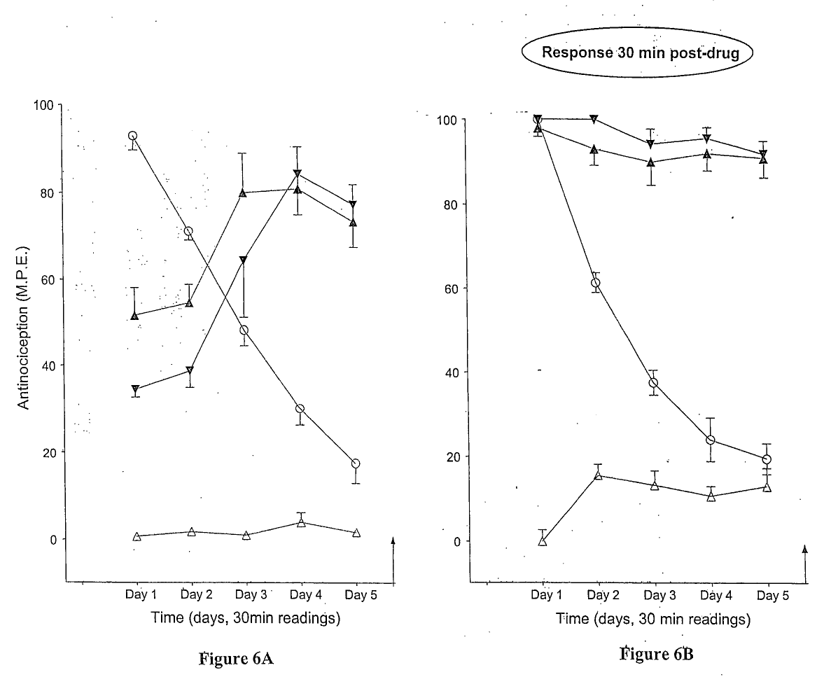

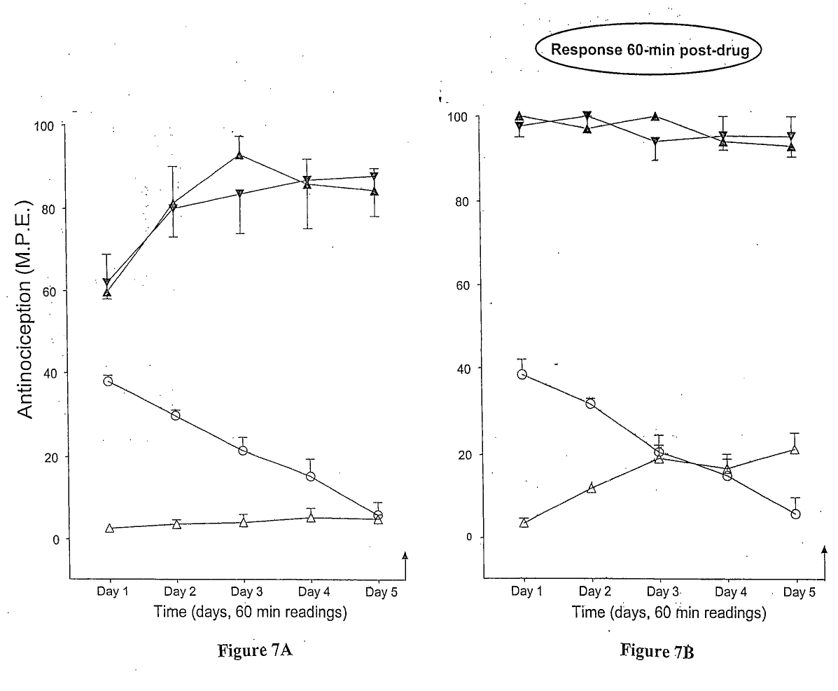

To investigate the effects of atipemazole on the development of chronic tolerance to intrathecal morphine, the opioid receptor agonist was delivered in combination with a fixed dose of atipemazole and nociceptive testing was performed daily. Cumulative dose-response curves for the acute intrathecal morphine were generated on day 6, as described above. The actions of atipemazole were assessed on the daily decline in magnitude of the morphine analgesia and on the morphine potency (i.e. ED₅₀ value) The effects of spinal atipemazole at ultra-low doses of 0.08 and 0.8 ng on chronic morphine tolerance induced by daily opioid administration are shown in FIGS. 6A and 6B and FIGS. 7A and 7B. The data represented in FIGS. 6 and 7 represent response measurements at 30 minutes (FIGS. 6A and 6B) and at 60 minutes (FIGS. 7A and 7B) after daily drug administration. As shown in FIGS. 6A and 6B, 30 minutes after administration of spinal morphine (15 μg), the analgesic response was at a maximal level on day 1. With daily drug administration, the magnitude of effect progressively declined towards baseline value by day 5. Injection of atipemazole with morphine delayed or inhibited this decline in both tests. Interestingly, the combination initially lowered the morphine effect in the tail flick test (FIGS. 6A and 7A), but this decrease was not maintained and the response to the combination exceeded the response to morphine at conclusion of the test period (day 5). In the paw pressure test (FIGS. 6B and 7B), however, the response to the atipemazole morphine combination was sustained at maximal level for the entire 5 day test period.

Measurement of the response taken at 60 minutes post injection (FIGS. 7A and 7B) showed that the effect of morphine at this time point was very much reduced in both tests. However, response to a combination therapy of the present invention comprising an ultra-low dose of atipemazole and morphine at this time point was maintained at or near maximal level in both tests. Thus, administering an alpha-2 receptor antagonist at an ultra-low dose to a subject chronically administered an opioid receptor agonist very effectively arrested the decline of opioid effect.

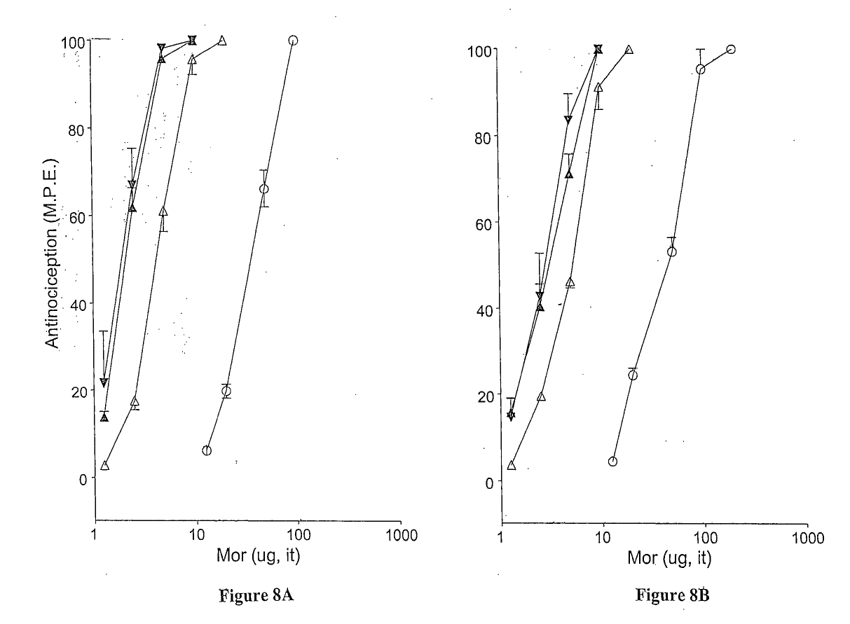

The cumulative dose-response curves for the action of morphine in the treatment groups represented in FIGS. 7A and 7B are shown in FIGS. 8A and 8B, respectively. These curves were derived on day 6, i.e. 24 hours after cessation of the 5 day chronic drug treatment. As was observed earlier, chronic morphine treatment produced a rightward shift in the dose-response curve, indicative of tolerance. Treatment with the exemplary atipemazole-morphine combination of the present invention prevented this rightward shift, a response indicative of blockade of tolerance.

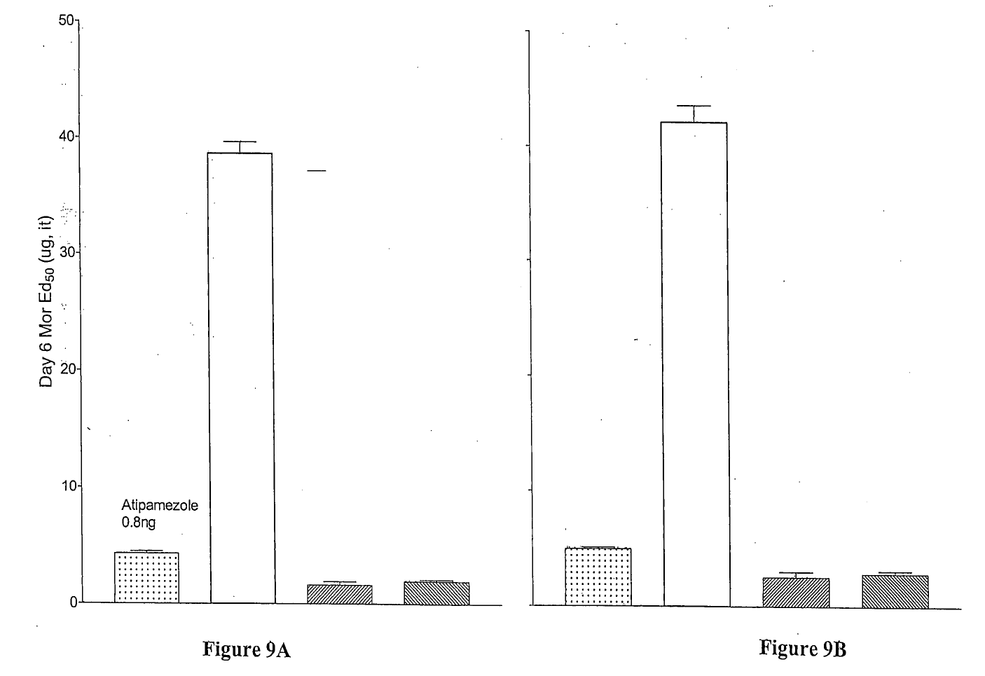

FIGS. 9A and 9B show ED₅₀ values derived from the cumulative dose-response curves presented in FIGS. 8A and 8B, respectively. The ED₅₀ values for morphine in the control group (that had received atipemazole alone) were approximately 5 μg. These were no different from those in the saline group. Chronic treatment with morphine produced nearly an 8-fold increase in the ED₅₀ values in both tests. This increase was completely prevented by introduction of atipemazole with morphine. Thus, an ultra-low dose of an alpha-2 receptor antagonist such as atipemazole clearly prevented the loss of potency in an opioid receptor agonist such as morphine that occurs with chronic administration and which signifies the induction of chronic tolerance. Accordingly, this ability to prevent loss in potency is also indicative of the combination therapies of the present invention inhibiting chronic tolerance of opioid receptor agonist therapy.

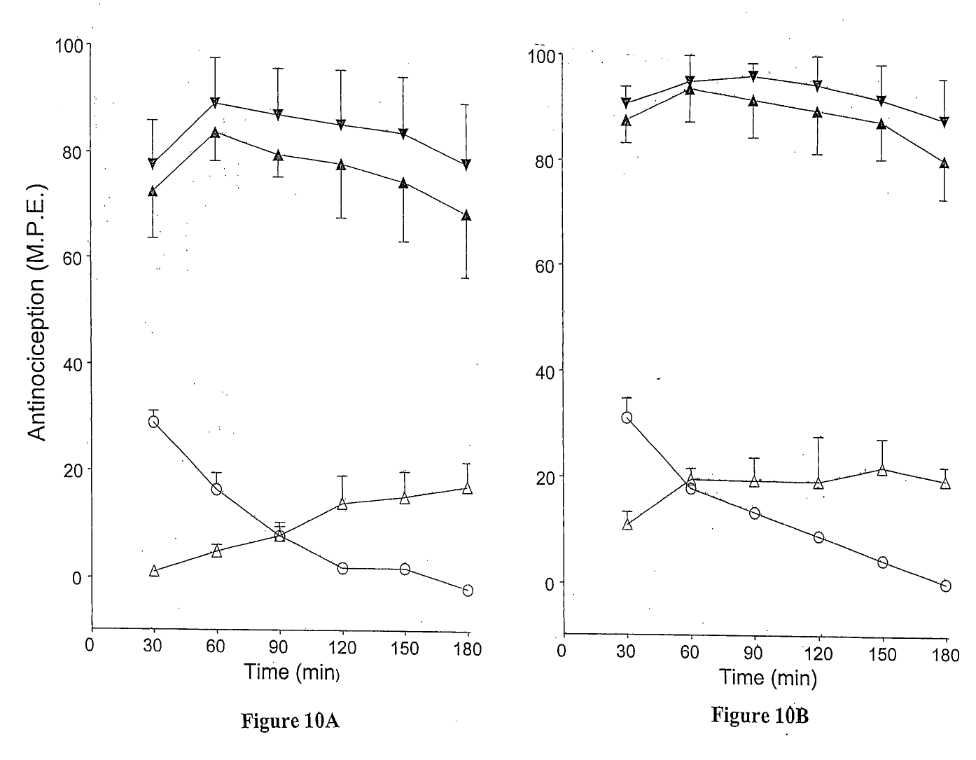

FIGS. 10A and 10B illustrate the time course of the analgesic responses produced by the atipemazole-morphine combination at conclusion of the chronic treatment period (day 5). As shown, the effect of morphine alone on day 5 was drastically reduced, but the response to the exemplary combination therapy of the present invention was maintained at a high level over the entire test period. Thus, both the peak effect and duration of the response elicited by the alpha-2 receptor antagonist and opioid receptor agonist combination therapy of the present invention exceeded the opioid receptor agonist effect.

Accordingly, as shown by these experiments, combination therapies of the present invention, wherein an ultra-low dose of an alpha-2 receptor antagonist is administered in combination with an opioid receptor agonist, blocks the progressive decline of analgesia following repeated opioid receptor agonist administration, prevents the rightward shift in the opioid receptor agonist dose-response curve obtained post chronic opioid exposure, and blocks the loss of drug potency (i.e. the increase in the ED₅₀ value of the opioid receptor agonist occurring post repeated treatment). Thus, these combination therapies of the present invention are useful in pain management in a subject.

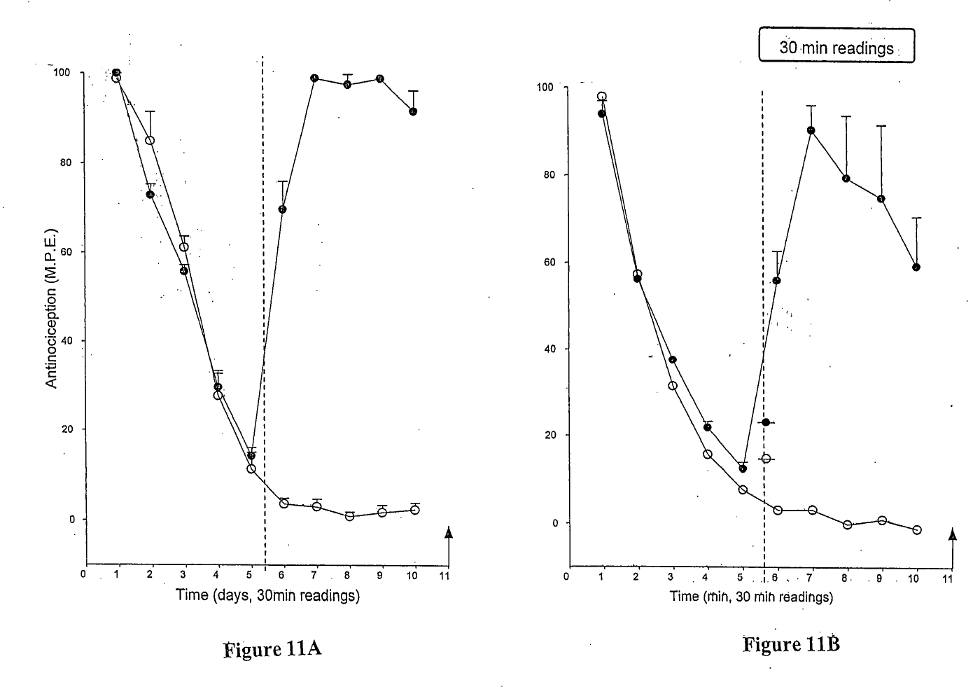

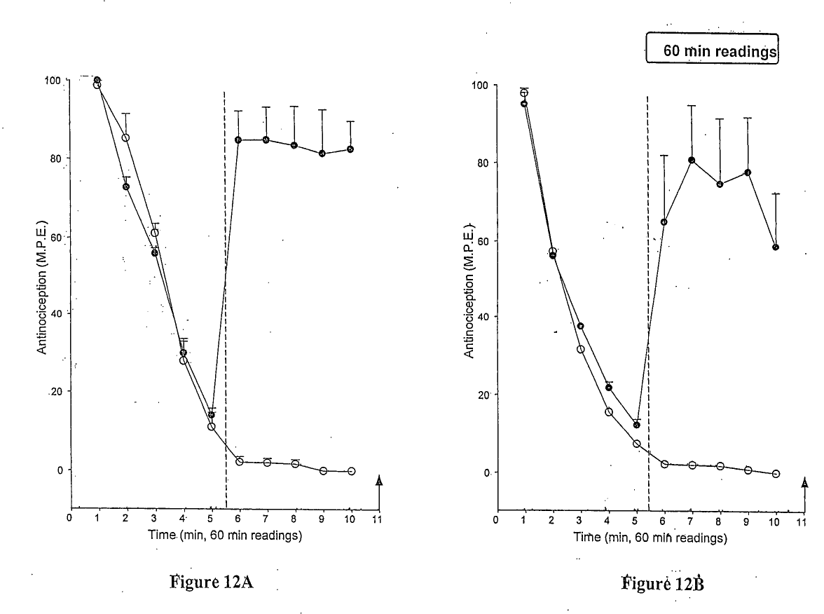

The ability of ultra-low doses of atipemazole to restore the potency of morphine in animals already tolerant to the analgesic action of the opioid receptor agonist was also demonstrated. In these experiments, an ultra-low dose (0.8 ng) of atipemazole was co-administered with morphine to animals made tolerant to opioid receptor agonists by chronic opioid receptor agonist treatment. The effects of atipemazole on established tolerance are illustrated in FIGS. 11A and 11B which depict nociception testing at 30 minutes post daily injection and in FIGS. 12A and 12B which depict nociception testing at 60 minutes post daily injection. As shown in FIGS. 11A and 11B, daily treatment with morphine resulted in progressive decline of the analgesic response in the tail flick and paw-pressure test, the response reaching near baseline value by day 5. Continuation of morphine on day 6 through day 10 maintained the analgesic response at this value. However, administration morphine with addition of atipemazole on day 6 produced a dramatic restoration of the response to morphine that approximated the original morphine response on day 1 and that remained significantly above baseline levels. Measurements of nociception taken at 60 minutes post daily injection (FIGS. 12A and B) revealed a similar profile of activity upon administration of an alpha-2 receptor antagonist with the opioid receptor agonist.

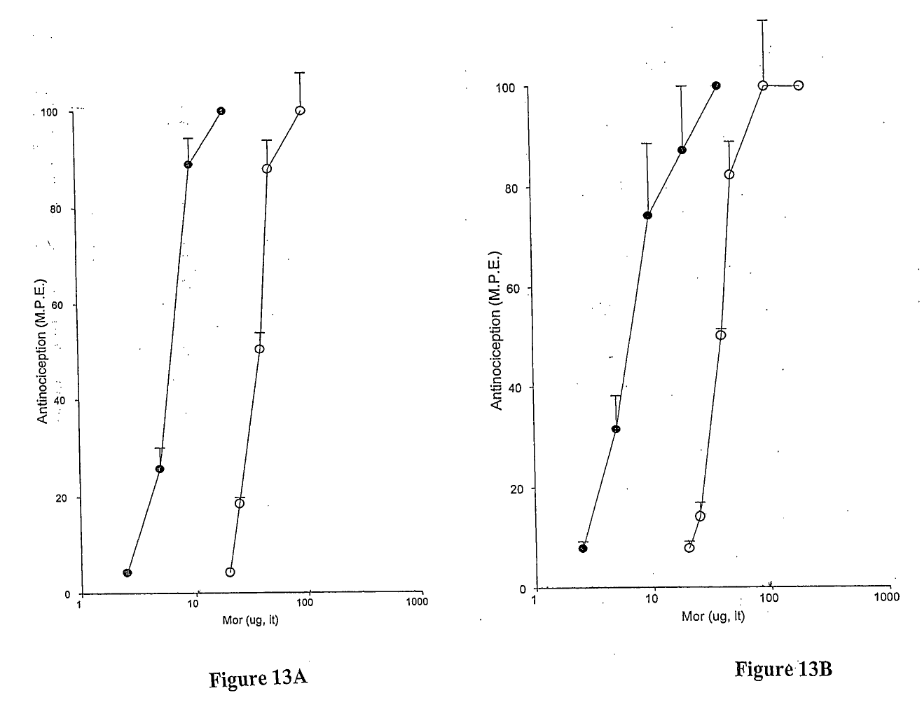

FIGS. 13A and B shows the cumulative dose-response curves for intrathecal morphine obtained in the two animal groups represented in FIGS. 12A and 12B. As shown by these Figures, in animals that had received morphine alone for 10 day period, the acute morphine dose-response curve was displaced to the right of the curve obtained in the group that had received morphine and atipemazole for the same period. The ability of atipemazole to produce a leftward shift is indicative of administration of an alpha-2 receptor antagonist restoring opioid receptor agonist potency.

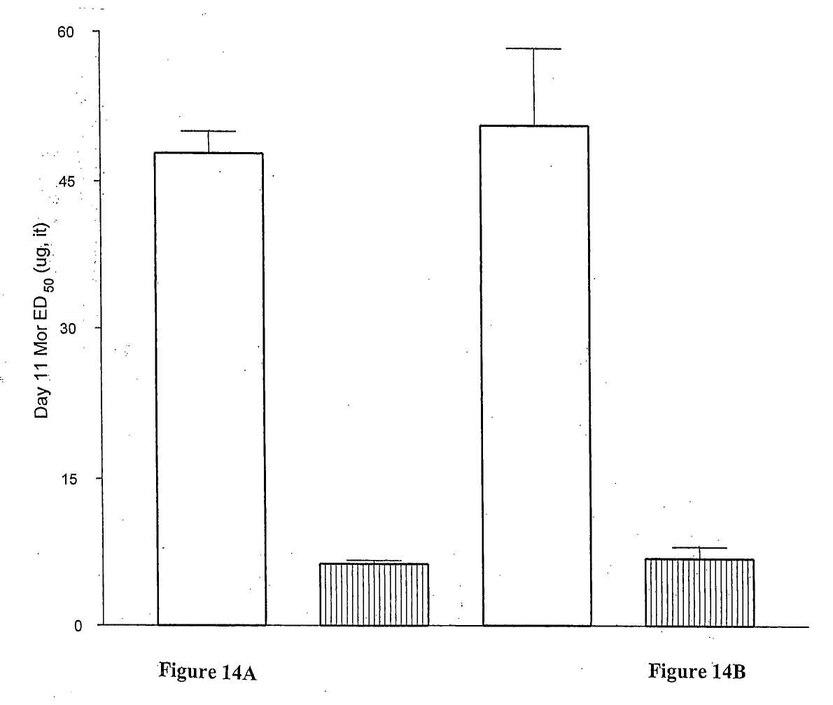

The morphine ED₅₀ values shown in FIGS. 14A and 14B, which were derived from the dose-response curves represented in FIGS. 13A and 13B, provide further quantitative evidence of this reversal of opioid receptor agonist tolerance by administration of an alpha-2 receptor antagonist at an ultra-low dose. The group of animals receiving chronic morphine alone exhibited ED₅₀ values approximating 47 and 48 μg in the tail flick and paw pressure test (unfilled bars). In contrast, the group receiving morphine with atipemazole showed ED₅₀ values approximating 6 and 8 μg. Thus, in animals unresponsive to the analgesic effects of morphine following chronic opioid receptor agonist exposure, the addition of atipemazole to the opioid receptor agonist restored its potency. The results demonstrate that administration of an alpha-2 receptor antagonist such as atipemazole actually reverses established tolerance to morphine analgesia.

As will be understood by the skilled artisan upon reading this disclosure, the present invention is not limited to the specific examples of potentiating opioid receptor agonist effects and inhibiting and/or reversing tolerance set forth herein, but rather, the invention should be construed and understood to include any combination of an opioid receptor agonist and alpha-2 receptor antagonist wherein such combination has the ability to potentiate the effect of the opioid receptor agonist as compared to the effect of the opioid receptor agonist when used alone or to inhibit and/or reverse tolerance to an opioid receptor agonist therapy. Based on the teachings set forth in extensive detail elsewhere herein, the skilled artisan will understand how to identify such opioid receptor agonists, alpha-2 receptor antagonists, and combinations thereof, as well as the concentrations of opioid receptor agonists and alpha-2 receptor antagonists to use in such a combination useful in the present invention.

As demonstrated herein, opioid receptor agonists and alpha-2 receptor antagonists can be administered, for example, epidurally or intrathecally. Further, as both morphine and atipemazole are know to be effective by systemic administration, i.e. orally or parenterally, it is expected that these therapeutic compounds will be effective following systemic administration as well. Accordingly, the combination therapies of the invention may be administered systemically or locally, and by any suitable route such as oral, buccal, sublingual, transdermal, subcutaneous, intraocular, intravenous, intramuscular or intraperitoneal administration, and the like (e.g., by injection) or via inhalation. Preferably, the opioid receptor agonist and alpha-2 receptor antagonist are administered simultaneously via the same route of administration. However, it is expected that administration of the compounds separately, via the same route or different route of administration, within a time frame during which each therapeutic compound remains active, will also be effective in pain management as well as in alleviating tolerance to the opioid receptor agonist. Further, as demonstrated herein, administration of an alpha-2 receptor antagonist to a subject already receiving opioid receptor agonist treatment reverses any tolerance to the opioid receptor agonist and restores analgesic potency of the opioid receptor agonist. Thus, treatment with the opioid receptor agonist and alpha-2 receptor antagonist in the combination therapy of the present invention need not begin at the same time. Instead, administration of the alpha-2 receptor antagonist may begin several days, weeks, months or more after treatment with the opioid receptor agonist. Alternatively, administration of the alpha-2 receptor antagonist may begin several days, weeks, months or more before treatment with the opioid receptor agonist.

Accordingly, for purposes of the present invention, the therapeutic compounds, namely the opioid receptor agonist and the alpha-2 receptor antagonist, can be administered together in a single pharmaceutically acceptable vehicle or separately, each in their own pharmaceutically acceptable vehicle.

As used herein, the term “therapeutic compound” is meant to refer to an opioid receptor agonist and/or an alpha-2 receptor antagonist.

As used herein “pharmaceutically acceptable vehicle” includes any and all solvents, excipients, dispersion media, coatings, antibacterial and antifungal agents, isotonic and absorption delaying agents, and the like which are compatible with the activity of the therapeutic compound and are physiologically acceptable to a subject. An example of a pharmaceutically acceptable vehicle is buffered normal saline (0.15 M NaCl). The use of such media and agents for pharmaceutically active substances is well known in the art. Except insofar as any conventional media or agent is incompatible with the therapeutic compound, use thereof in the compositions suitable for pharmaceutical administration is contemplated. Supplementary active compounds can also be incorporated into the compositions.

Carrier or substituent moieties useful in the present invention may also include moieties which allow the therapeutic compound to be selectively delivered to a target organ. For example, delivery of the therapeutic compound to the brain may be enhanced by a carrier moiety using either active or passive transport (a “targeting moiety”). Illustratively, the carrier molecule may be a redox moiety, as described in, for example, U.S. Pat. Nos. 4,540,654 and 5,389,623, both to Bodor. These patents disclose drugs linked to dihydropyridine moieties which can enter the brain, where they are oxidized to a charged pyridinium species which is trapped in the brain. Thus drugs linked to these moieties accumulate in the brain. Other carrier moieties include compounds, such as amino acids or thyroxine, which can be passively or actively transported in vivo. Such a carrier moiety can be metabolically removed in vivo, or can remain intact as part of an active compound.

Structural mimics of amino acids (and other actively transported moieties) including peptidomimetics, are also useful in the invention. As used herein, the term “peptidomimetic” is intended to include peptide analogues which serve as appropriate substitutes for peptides in interactions with, for example, receptors and enzymes. The peptidomimetic must possess not only affinity, but also efficacy and substrate function. That is, a peptidomimetic exhibits functions of a peptide, without restriction of structure to amino acid constituents. Peptidomimetics, methods for their preparation and use are described in Morgan et al. (1989)(“Approaches to the discovery of non-peptide ligands for peptide receptors and peptidases,” In Annual Reports in Medicinal Chemistry (Virick, F. J., ed.), Academic Press, San Diego, Calif., pp. 243-253), the contents of which are incorporated herein by reference. Many targeting moieties are known, and include, for example, asialoglycoproteins (see e.g., Wu, U.S. Pat. No. 5,166,320) and other ligands which are transported into cells via receptor-mediated endocytosis (see below for further examples of targeting moieties which may be covalently or non-covalently bound to a target molecule).

The term “subject” as used herein is intended to include living organisms in which pain to be treated can occur. Examples of subjects include mammals such as humans, apes, monkeys, cows, sheep, goats, dogs, cats, mice, rats, and transgenic species thereof. As would be apparent to a person of skill in the art, the animal subjects employed in the working examples set forth below are reasonable models for human subjects with respect to the tissues and biochemical pathways in question, and consequently the methods, therapeutic compounds and pharmaceutical compositions directed to same. As evidenced by Mordenti (J. Pharm. Sci. 1986 75 (11):1028-40) and similar articles, dosage forms for animals such as, for example, rats can be and are widely used directly to establish dosage levels in therapeutic applications in higher mammals, including humans. In particular, the biochemical cascade initiated by many physiological processes and conditions is generally accepted to be identical in mammalian species (see, e.g., Mattson and Scheff, Neurotrauma 1994 11 (1):3-33; Higashi et al. Neuropathol. Appl. Neurobiol. 1995 21:480-483). In light of this, pharmacological agents that are efficacious in animal models such as those described herein are believed to be predictive of clinical efficacy in humans, after appropriate adjustment of dosage.

Depending on the route of administration, the therapeutic compound may be coated in a material to protect the compound from the action of acids, enzymes and other natural conditions which may inactivate the compound. Insofar as the invention provides a combination therapy in which two therapeutic compounds are administered, each of the two compounds may be administered by the same route or by a different route. Also, the compounds may be administered either at the same time (i.e., simultaneously) or each at different times. In some treatment regimes it may be beneficial to administer one of the compounds more or less frequently than the other.

The compounds of the invention can be formulated to ensure proper distribution in vivo. For example, the blood-brain barrier (BBB) excludes many highly hydrophilic compounds. To ensure that the therapeutic compounds of the invention cross the BBB, they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs (“targeting moieties”), thus providing targeted drug delivery (see, e.g., Ranade, V. V. J. Clin. Pharmacol. 1989 29 (8):685-94). Exemplary targeting moieties include folate and biotin (see, e.g., U.S. Pat. No. 5,416,016 to Low et al.); mannosides (Umezawa et al. Biochem. Biophys. Res. Commun. 1988 153 (3):1038-44; antibodies (Bloeman et al. FEBS Lett. 1995 357:140; Owais et al. Antimicrob. Agents Chemother. 1995 39 (1):180-4); and surfactant protein A receptor (Briscoe et al. Am. J. Physiol. 1995 268 (3 Pt 1):L374-80). In a preferred embodiment, the therapeutic compounds of the invention are formulated in liposomes; in a more preferred embodiment, the liposomes include a targeting moiety.

Delivery and in vivo distribution can also be affected by alteration of an anionic group of compounds of the invention. For example, anionic groups such as phosphonate or carboxylate can be esterified to provide compounds with desirable pharmacokinetic, pharmacodynamic, biodistributive, or other properties.

To administer a therapeutic compound by other than parenteral administration, it may be necessary to coat the compound with, or co-administer the compound with, a material to prevent its inactivation. For example, the therapeutic compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al. Prog. Clin. Biol. Res. 1984 146:429-34).

The therapeutic compound may also be administered parenterally (e.g., intramuscularly, intravenously, intraperitoneally, intraspinally, intrathecally, or intracerebrally). Dispersions can be prepared in glycerol, liquid polyethylene glycols, and mixtures thereof and in oils. Under ordinary conditions of storage and use, these preparations may contain a preservative to prevent the growth of microorganisms. Pharmaceutical compositions suitable for injectable use include sterile aqueous solutions (where water soluble) or dispersions and sterile powders for the extemporaneous preparation of sterile injectable solutions or dispersions. In all cases, the composition must be sterile and must be fluid to the extent that easy syringability exists. It must be stable under the conditions of manufacture and storage and must be preserved against the contaminating action of microorganisms such as bacteria and fungi. The vehicle can be a solvent or dispersion medium containing, for example, water, ethanol, polyol (for example, glycerol, propylene glycol, liquid polyethylene glycol, and the like), suitable mixtures thereof, and oils (e.g.,vegetable oil). The proper fluidity can be maintained, for example, by the use of a coating such as lecithin, by the maintenance of the required particle size in the case of dispersion, and by the use of surfactants.

Prevention of the action of microorganisms can be achieved by various antibacterial and antifungal agents, for example, parabens, chlorobutanol, phenol, ascorbic acid, thimerosal, and the like. In some cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic compound in the required amount in an appropriate solvent with one or a combination of ingredients enumerated above, as required, followed by filter sterilization. Generally, dispersions are prepared by incorporating the therapeutic compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yield a powder of the active ingredient (i.e., the therapeutic compound) optionally plus any additional desired ingredient from a previously sterile-filtered solution thereof.

Solid dosage forms for oral administration include ingestible capsules, tablets, pills, lollipops, powders, granules, elixirs, suspensions, syrups, wafers, buccal tablets, troches, and the like. In such solid dosage forms the active compound is mixed with at least one inert, pharmaceutically acceptable excipient or diluent or assimilable edible carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof, or incorporated directly into the subject's diet. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like. The percentage of the therapeutic compound in the compositions and preparations may, of course, be varied. The amount of the therapeutic compound in such therapeutically useful compositions is such that a suitable dosage will be obtained.

The solid dosage forms of tablets, dragees, capsules, pills, and granules can be prepared with coatings and shells such as enteric coatings and other coatings well-known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, ground nut corn, germ olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Therapeutic compounds can be administered in time-release or depot form, to obtain sustained release of the therapeutic compounds over time. The therapeutic compounds of the invention can also be administered transdermally (e.g., by providing the therapeutic compound, with a suitable carrier, in patch form).

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of neurological conditions in subjects.

Therapeutic compounds according to the invention are administered at a therapeutically effective dosage sufficient to achieve the desired therapeutic effect of the opioid receptor agonist, e.g. to mitigate pain and/or to effect analgesia in a subject, to suppress coughs, to reduce and/or prevent diarrhea, to treat pulmonary edema or to alleviate addiction to opioid receptor agonists. For example, if the desired therapeutic effect is analgesia, the “therapeutically effective dosage” mitigates pain by about 25%, preferably by about 50%, even more preferably by about 75%, and still more preferably by about 100% relative to untreated subjects. Actual dosage levels of active ingredients in the pharmaceutical compositions of this invention may be varied so as to obtain an amount of the active compound(s) that is effective to achieve and maintain the desired therapeutic response for a particular subject, composition, and mode of administration. The selected dosage level will depend upon the activity of the particular compound, the route of administration, frequency of administration, the severity of the condition being treated, the condition and prior medical history of the subject being treated, the age, sex, weight and genetic profile of the subject, and the ability of the therapeutic compound to produce the desired therapeutic effect in the subject. Dosage regimens can be adjusted to provide the optimum therapeutic response. For example, several divided doses may be administered daily or the dose may be proportionally reduced as indicated by the exigencies of the therapeutic situation.

However, it is well known within the medical art to determine the proper dose for a particular patient by the dose titration method. In this method, the patient is started with a dose of the drug compound at a level lower than that required to achieve the desired therapeutic effect. The dose is then gradually increased until the desired effect is achieved. Starting dosage levels for an already commercially available therapeutic agent of the classes discussed above can be derived from the information already available on the dosages employed. Also, dosages are routinely determined through preclinical ADME toxicology studies and subsequent clinical trials as required by the FDA or equivalent agency. The ability of an opioid receptor agonist to produce the desired therapeutic effect may be demonstrated in various well known models for the various conditions treated with these therapeutic compounds. For example, mitigation of pain can be evaluated in model systems that may be predictive of efficacy in mitigating pain in human diseases and trauma, such as animal model systems known in the art (including, e.g., the models described herein).

Compounds of the invention may be formulated in such a way as to reduce the potential for abuse of the compound. For example, a compound may be combined with one or more other agents that prevent or complicate separation of the compound therefrom.

The following nonlimiting examples are provided to further illustrate the present invention.

### EXAMPLES

### Example 1

### Animals

Experiments were conducted using adult male Sprague-Dawley rats (Charles River, St. Constant, QC, Canada) weighing between 200-250 grams. Animals were housed individually in standard laboratory cages, maintained on a 12-hour light/dark cycle, and provided with food and water ad libitum. The surgical placement of chronic indwelling intrathecal catheters (polyethylene PE 10 tubing, 7.5 cm) into the spinal subarachnoid space was made under 4% halothane anesthesia, using the method of Yaksh and Rudy Physiol. Behav. 1976 7:1032-1036). Specifically, the anesthetized animal was placed prone in a stereotaxic frame, a small incision made at the back of the neck, and the atlanto-occipital membrane overlying the cisterna magna was exposed and punctured with a blunt needle. The catheter was inserted through the cisternal opening and slowly advanced caudally to position its tip at the lumbar enlargement. The rostral end of the catheter was exteriorized at the top of the head and the wound closed with sutures. Animals were allowed 3-4 days recovery from surgery and only those free from neurological deficits, such as the hindlimb or forelimb paralysis or gross motor dysfunction, were included in the study. All drugs were injected intrathecally as solutions dissolved in physiological saline (0.9%) through the exteriorized portion of the catheter at a volume of 10 μl, followed by a 10 μl volume of 0.9% saline to flush the catheter.

### Example 2

### Assessment of Nociception

The response to brief nociceptive stimuli was tested using two tests: the tail flick test and the paw pressure test.

The tail flick test (D'amour & Smith, J. Pharmacol. Exp. Ther. 1941 72:74-79) was used to measure the response to a thermal nociceptive stimulus. Radiant heat was applied to the distal third of the animal's tail and the response latency for tail withdrawal from the source was recorded using an analgesia meter (Owen et al., J. Pharmacol. Methods 1981 6:33-37)). The stimulus intensity was adjusted to yield baseline response latencies between 2-3 seconds. To minimize tail damage, a cutoff of 10 seconds was used as an indicator of maximum antinociception.

The paw pressure test (Loomis et al., Pharm. Biochem. 1987 26:131-139) was used to measure the response to a mechanical nociceptive stimulus. Pressure was applied to the dorsal surface of the hind paw using an inverted air-filled syringe connected to a gauge and the value at which the animal withdrew its paw was recorded. A maximum cutoff pressure of 300 mmHg was used to avoid tissue damage. Previous experience has established that there is no significant interaction between the tail flick and paw pressure tests (Loomis et al., Can. J. Physiol. Pharmacol. 1985 63:656-662).

### Example 3

### Determination of Inhibition of Clonidine and/or Morphine Analgesia by Alpha-2 Receptor Antagonists

The effects of atipemazole, yohimbine, idazoxan and mirtazapine were tested on the acute analgesic action of spinal clonidine to establish that each of these drugs act as alpha-2 receptor antagonists. A single injection of clonidine was administered intrathecally and the response measured in the tail flick and paw pressure test. In subsequent tests, clonidine was delivered in combination with 1, 5 or 10 μg atipemazole, 30 μg yohimbine, 10 μg idazoxan or 2 μg mirtazapine. Following drug administration, nociceptive testing was performed every 10 minutes for the first 60 minutes and every 30 minutes for the following 120-150 minute period. Results for atipemazole are depicted in FIG. 1A (tail flick) and FIG. 1B (paw pressure). Results for yohimbine are depicted in FIG. 15A (tail flick) and FIG. 15B (paw pressure). Results for idazoxan are depicted in FIG. 28A (tail flick) and FIG. 28B (paw pressure). Results for mirtazapine are depicted in FIG. 21A (tail flick) and FIG. 21B (paw pressure). Similar experiments were performed with yohimbine at 30 μg in combination with morphine. See FIG. 16A (tail flick) and FIG. 16B (paw pressure).

### Example 4

### Reversal of the Pre-existing Morphine Analgesic Tolerance by Ultra-low Dose Atipemazole

Chronic tolerance was induced in rats by intrathecal injection of morphine (15 μg) once daily for 5-days. Animals were divided into two groups and nociceptive testing was performed 30 minutes and 60 minutes after the daily drug injection using the tail flick and paw pressure test. On day 6, one group continued on this morphine dose for additional 5 days whereas the other group received morphine in combination with a low dose of atipemazole (0.8 ng) for the same period. Nociception was assessed on a daily basis as described above. On day 11, cumulative dose-response curves for the action of acute intrathecal morphine were generated to obtain index of morphine potency (ED₅₀ values)

### Example 5

### Data Analysis

For the in vivo studies, tail flick and paw pressure values were converted to a maximum percentage effect (M.P.E.): M.P.E.=100×[post-drug response−baseline response]/[maximum response−baseline response]. Data represented in the figures are expressed as mean (±S.E.M.). The ED₅₀ values were determined using a non-linear regression analysis (Prism 2, GraphPad Software Inc., San Diego, Calif., USA). Statistical significance (p<0.05, 0.01. or 0.001) was determined using a one-way analysis of variance followed by a Student Newman-Keuls post hoc test for multiple comparisons between groups.

What is claimed is:

1. A composition comprising an opioid receptor agonist in an amount effective to produce a therapeutic effect and an alpha-2 receptor antagonist in an amount effective to potentiate, but not antagonize, a therapeutic effect of the opioid receptor agonist.

2. The composition of claim 1 wherein the opioid receptor agonist is an opioid.

3. The composition of claim 1 wherein the opioid receptor agonist is selected from the group consisting of morphine, oxycodone, oxymorphone, hydromorphone, mepridine, methadone, fentanyl, sufentanil, alfentanil, remifentanil, carfentanil, lofentanil, codeine, hydrocodone, levorphanol, tramadol, D-Pen2, D-Pen5-enkephalin (DPDPE), U50, 488H (trans-3,4-dichloro-N-methyl-N-[2-pyrrolindinyl]-cyclohexanyl)-benzeneacetamide, endorphins, dynorphins, enkephalins, diamorphine (heroin), dihydrocodeine, nicomorphine, levomethadyl acetate hydrochloride (LAAM), ketobemidone, propoxyphene, dextropropoxyphene, dextromoramide, bezitramide, piritramide, pentazocine, phenazocine, buprenorphine, butorphanol, nalbufine or nalbuphine, dezocine, etorphine, tilidine, loperamide, diphenoxylate, paregoric and nalorphine.

4. The composition of claim 1 wherein the alpha-2 receptor antagonist is selected from the group consisting of atipemazole (or atipamezol), fipamazole (fluorinated derivative of atipemazole), mirtazepine (or mirtazapine), eferoxan, idozoxan (or idazoxan), Rx821002 (2-methoxy-idozoxan), rauwolscine, MK 912, SKF 86466, SKF 1563 and yohimbine.

5. The composition of claim 1 wherein the alpha-2 receptor antagonist is selected from the group consisting of venlafaxine, doxazosin, phentolamine, dihydroergotamine, ergotamine, phenothiazines, phenoxybenzamine, piperoxane, prazosin, tamsulosin, terazosin, and tolazoline.

6. The composition of claim 1 wherein the opioid receptor agonist is morphine and the alpha-2 receptor antagonist is atipemazole (or atipamezol), mirtazepine (or mirtazapine), idozoxan (or idazoxan) or yohimbine.

7. The composition of claim 1 wherein the opioid receptor agonist is oxycodone and the alpha-2 receptor antagonist is atipemazole (or atipamezol), mirtazepine (or mirtazapine), idozoxan (or idazoxan) or yohimbine.

8. A method for potentiating a therapeutic effect of an opioid receptor agonist in a subject, the method comprising administering an opioid receptor agonist to the subject and administering an alpha-2 receptor antagonist to the subject in an amount effective to potentiate, but not antagonize, the therapeutic effect of the opioid receptor agonist.

9. The method of claim 8 wherein the opioid receptor agonist is an opioid.

10. The method of claim 8 wherein the opioid receptor agonist is selected from the group consisting of morphine, oxycodone, oxymorphone, hydromorphone, mepridine, methadone, fentanyl, sufentanil, alfentanil, remifentanil, carfentanil, lofentanil, codeine, hydrocodone, levorphanol, tramadol, D-Pen2, D-Pen5-enkephalin (DPDPE), U50, 488H (trans-3,4-dichloro-N-methyl-N-[2-pyrrolindinyl]-cyclohexanyl)-benzeneacetamide, endorphins, dynorphins, enkephalins, diamorphine (heroin), dihydrocodeine, nicomorphine, levomethadyl acetate hydrochloride (LAAM), ketobemidone, propoxyphene, dextropropoxyphene, dextromoramide, bezitramide, piritramide, pentazocine, phenazocine, buprenorphine, butorphanol, nalbufine or nalbuphine, dezocine, etorphine, tilidine, loperamide, diphenoxylate, paregoric and nalorphine.

11. The method of claim 8 wherein the alpha-2 receptor antagonist is selected from the group consisting of atipemazole (or atipamezol), fipamazole (fluorinated derivative of atipemazole), mirtazepine (or mirtazapine), eferoxan, idozoxan (or idazoxan), Rx821002 (2-methoxy-idozoxan), rauwolscine, MK 912, SKF 86466, SKF 1563 and yohimbine.

12. The method of claim 8 wherein the alpha-2 receptor antagonist is selected from the group consisting of venlafaxine, doxazosin, phentolamine, dihydroergotamine, ergotamine, phenothiazines, phenoxybenzamine, piperoxane, prazosin, tamsulosin, terazosin, and tolazoline.

13. The method of claim 8 wherein the therapeutic effect of the opioid receptor agonist is potentiated without substantial undesirable side effects.

14. A method for potentiating a therapeutic effect of an endogenous opioid receptor agonist in a subject, the method comprising administering to the subject an alpha-2 receptor antagonist, in an amount effective to potentiate, but not antagonize the therapeutic effect of the endogenous opioid receptor agonist.

15. The method of claim 14 wherein the endogenous opioid receptor agonist is selected from the group consisting of beta-endorphins, enkephalins and dynorphins.

16. The method of claim 14 wherein the alpha-2 receptor antagonist is selected from the group consisting of atipemazole (or atipamezol), fipamazole (fluorinated derivative of atipemazole), mirtazepine (or mirtazapine), eferoxan, idozoxan (or idazoxan), Rx821002 (2-methoxy-idozoxan), rauwolscine, MK 912, SKF 86466, SKF 1563 and yohimbine.

17. The method of claim 14 wherein the alpha-2 receptor antagonist is selected from the group consisting of venlafaxine, doxazosin, phentolamine, dihydroergotamine, ergotamine, phenothiazines, phenoxybenzamine, piperoxane, prazosin, tamsulosin, terazosin, and tolazoline.

18. A method for inhibiting development of acute tolerance to a therapeutic effect of an opioid receptor agonist in a subject, the method comprising administering the opioid receptor agonist to the subject and administering an alpha-2 receptor antagonist to the subject in an amount effective to potentiate, but not antagonize, the therapeutic effect of the opioid receptor agonist.

19. The method of claim 18 wherein the opioid receptor agonist is an opioid.

20. The method of claim 18 wherein the opioid receptor agonist is selected from the group consisting of morphine, oxycodone, oxymorphone, hydromorphone, mepridine, methadone, fentanyl, sufentanil, alfentanil, remifentanil, carfentanil, lofentanil, codeine, hydrocodone, levorphanol, tramadol, D-Pen2, D-Pen5-enkephalin (DPDPE), U50, 488H (trans-3,4-dichloro-N-methyl-N-[2-pyrrolindinyl]-cyclohexanyl)-benzeneacetamide, endorphins, dynorphins, enkephalins, diamorphine (heroin), dihydrocodeine, nicomorphine, levomethadyl acetate hydrochloride (LAAM), ketobemidone, propoxyphene, dextropropoxyphene, dextromoramide, bezitramide, piritramide, pentazocine, phenazocine, buprenorphine, butorphanol, nalbufine or nalbuphine, dezocine, etorphine, tilidine, loperamide, diphenoxylate, paregoric and nalorphine.

21. The method of claim 18 wherein the alpha-2 receptor antagonist is selected from the group consisting of atipemazole (or atipamezol), fipamazole (fluorinated derivative of atipemazole), mirtazepine (or mirtazapine), eferoxan, idozoxan (or idazoxan), Rx821002 (2-methoxy-idozoxan), rauwolscine, MK 912, SKF 86466, SKF 1563 and yohimbine.

22. The method of claim 18 wherein the alpha-2 receptor antagonist is selected from the group consisting of venlafaxine, doxazosin, phentolamine, dihydroergotamine, ergotamine, phenothiazines, phenoxybenzamine, piperoxane, prazosin, tamsulosin, terazosin, and tolazoline.

23. A method for inhibiting development of chronic tolerance to a therapeutic effect of an opioid receptor agonist in a subject, the method comprising administering the opioid receptor agonist to the subject and administering an alpha-2 receptor antagonist to the subject in an amount effective to potentiate, but not antagonize, the therapeutic effect of the opioid receptor agonist.

24. The method of claim 23 wherein the opioid receptor agonist is an opioid.

25. The method of claim 23 wherein the opioid receptor agonist is selected from the group consisting of morphine, oxycodone, oxymorphone, hydromorphone, mepridine, methadone, fentanyl, sufentanil, alfentanil, remifentanil, carfentanil, lofentanil, codeine, hydrocodone, levorphanol, tramadol, D-Pen2, D-Pen5-enkephalin (DPDPE), U50, 488H (trans-3,4-dichloro-N-methyl-N-[2-pyrrolindinyl]-cyclohexanyl)-benzeneacetamide, endorphins, dynorphins, enkephalins, diamorphine (heroin), dihydrocodeine, nicomorphine, levomethadyl acetate hydrochloride (LAAM), ketobemidone, propoxyphene, dextropropoxyphene, dextromoramide, bezitramide, piritramide, pentazocine, phenazocine, buprenorphine, butorphanol, nalbufine or nalbuphine, dezocine, etorphine, tilidine, loperamide, diphenoxylate, paregoric and nalorphine.

26. The method of claim 23 wherein the alpha-2 receptor antagonist is selected from the group consisting of atipemazole (or atipamezol), fipamazole (fluorinated derivative of atipemazole), mirtazepine (or mirtazapine), eferoxan, idozoxan (or idazoxan), Rx821002 (2-methoxy-idozoxan), rauwolscine, MK 912, SKF 86466, SKF 1563 and yohimbine.

27. The method of claim 23 wherein the alpha-2 receptor antagonist is selected from the group consisting of venlafaxine, doxazosin, phentolamine, dihydroergotamine, ergotamine, phenothiazines, phenoxybenzamine, piperoxane, prazosin, tamsulosin, terazosin, and tolazoline.

28. A method for reversing tolerance to a therapeutic effect of an opioid receptor agonist or restoring a therapeutic effect of an opioid receptor agonist in a subject, the method comprising administering to the subject an alpha-2 receptor antagonist in an amount effective to potentiate, but not antagonize, the therapeutic effect of the opioid receptor agonist.

29. The method of claim 28 wherein the alpha-2 receptor antagonist is selected from the group consisting of atipemazole (or atipamezol), fipamazole (fluorinated derivative of atipemazole), mirtazepine (or mirtazapine), eferoxan, idozoxan (or idazoxan), Rx821002 (2-methoxy-idozoxan), rauwolscine, MK 912, SKF 86466, SKF 1563 and yohimbine.

30. The method of claim 28 wherein the alpha-2 receptor antagonist is selected from the group consisting of venlafaxine, doxazosin, phentolamine, dihydroergotamine, ergotamine, phenothiazines, phenoxybenzamine, piperoxane, prazosin, tamsulosin, terazosin, and tolazoline.

31. A method for treating a subject suffering from a condition treatable with an opioid receptor agonist, the method comprising administering an opioid receptor agonist to the subject in an amount effective to produce a therapeutic effect and administering an alpha-2 receptor antagonist to the subject in an amount effective to potentiate, but not antagonize, the therapeutic effect of the opioid receptor agonist.

32. The method of claim 31 wherein the opioid receptor agonist is an opioid.

33. The method of claim 31 wherein the opioid receptor agonist is selected from the group consisting of morphine, oxycodone, oxymorphone, hydromorphone, mepridine, methadone, fentanyl, sufentanil, alfentanil, remifentanil, carfentanil, lofentanil, codeine, hydrocodone, levorphanol, tramadol, D-Pen2, D-Pen5-enkephalin (DPDPE), U50, 488H (trans-3,4-dichloro-N-methyl-N-[2-pyrrolindinyl]-cyclohexanyl)-benzeneacetamide, endorphins, dynorphins, enkephalins, diamorphine (heroin), dihydrocodeine, nicomorphine, levomethadyl acetate hydrochloride (LAAM), ketobemidone, propoxyphene, dextropropoxyphene, dextromoramide, bezitramide, piritramide, pentazocine, phenazocine, buprenorphine, butorphanol, nalbufine or nalbuphine, dezocine, etorphine, tilidine, loperamide, diphenoxylate, paregoric and nalorphine.

34. The method of claim 31 wherein the alpha-2 receptor antagonist is selected from the group consisting of atipemazole (or atipamezol), fipamazole (fluorinated derivative of atipemazole), mirtazepine (or mirtazapine), eferoxan, idozoxan (or idazoxan), Rx821002 (2-methoxy-idozoxan), rauwolscine, MK 912, SKF 86466, SKF 1563 and yohimbine.

35. The method of claim 31 wherein the alpha-2 receptor antagonist is selected from the group consisting of venlafaxine, doxazosin, phentolamine, dihydroergotamine, ergotamine, phenothiazines, phenoxybenzamine, piperoxane, prazosin, tamsulosin, terazosin, and tolazoline.

36. The method of claim 31 wherein the subject is suffering from pain, coughing, diarrhea, pulmonary edema or addiction to an opioid receptor agonist.

37. The method of claim 36 wherein the pain is acute or chronic post-surgical pain, obstetrical pain, acute inflammatory pain, chronic inflammatory pain, pain associated with multiple sclerosis or cancer, pain associated with trauma, pain associated with migraines, neuropathic pain, central pain or a chronic pain syndrome of a non-malignant origin, or chronic back pain.

38. The method of claim 31 wherein the subject is treated for a condition treatable with an opioid receptor agonist without substantial undesirable side effects.

39. A method for treating a subject suffering from a condition treatable with an opioid receptor agonist comprising administering to a subject receiving opioid receptor agonist therapy an alpha-2 receptor antagonist in an amount effective to potentiate, but not antagonize the therapeutic effect of the opioid receptor agonist.

40. The method of claim 39 wherein the alpha-2 receptor antagonist is selected from the group consisting of atipemazole (or atipamezol), fipamazole (fluorinated derivative of atipemazole), mirtazepine (or mirtazapine), eferoxan, idozoxan (or idazoxan), Rx821002 (2-methoxy-idozoxan), rauwolscine, MK 912, SKF 86466, SKF 1563 and yohimbine.

41. The method of claim 39 wherein the alpha-2 receptor antagonist is selected from the group consisting of venlafaxine, doxazosin, phentolamine, dihydroergotamine, ergotamine, phenothiazines, phenoxybenzamine, piperoxane, prazosin, tamsulosin, terazosin, and tolazoline.

42. The method of claim 39 wherein the subject is suffering from pain, coughing, diarrhea, pulmonary edema or addiction to an opioid receptor agonist.

43. The method of claim 42 wherein the subject is suffering from acute or chronic post-surgical pain, obstetrical pain, acute inflammatory pain, chronic inflammatory pain, pain associated with multiple sclerosis or cancer, pain associated with trauma, pain associated with migraines, neuropathic pain, central pain or chronic pain syndrome of a non-malignant origin.

44. The method of claim 39 wherein the subject is treated for a condition treatable with an alpha-2 adrenergic receptor agonist without substantial undesirable side effects.
